(19) United States
(12) Patent Application Publication
(10) Pub. No.: US 2018/0353539 A1
(43) Pub. Date: Dec. 13, 2018

(54) METAL COMPLEXES AS PHARMACEUTICALS FOR TREATMENT AND PREVENTION OF CANCER AND INFLAMMATORY DISEASES

(71) Applicant: UNIVERSITY OF SOUTHERN CALIFORNIA, Los Angeles, CA (US)

(72) Inventors: BO HAN, TEMPLE CITY, CA (US); BA XUAN HOANG, CYPRESS, CA (US)

(21) Appl. No.: 16/006,008

(22) Filed: Jun. 12, 2018

Related U.S. Application Data
(60) Provisional application No. 62/518,050, filed on Jun. 12, 2017.

(51) Int. Cl. A61K 33/30; A61K 31/60; A61P 35/04; A61P 11/00; A61P 35/00; A61P 35/02; A61N 1/32

(52) U.S. Cl. CPC A61K 33/30; A61K 31/60; A61P 35/04; A61N 1/325; A61P 35/00; A61P 35/02; A61P 11/00

(57) ABSTRACT

A method for treating cancer, chronic obstructive pulmonary disease, asthma, diabetes, sickle cell disease, thalassemia, or any other inflammatory disease, and/or promoting healing thereafter includes a step of identifying a subject afflicted with cancer, chronic obstructive pulmonary disease, or an inflammatory disease. A therapeutically effective dose of a pharmaceutical composition is administered to the patient. The pharmaceutical composition includes at least one metal-containing compound. Characteristically, the at least one metal-containing compound includes a metal selected from the group consisting of zinc, copper, manganese, and combinations thereof.

### CROSS-REFERENCE TO RELATED APPLICATIONS

This application claims the benefit of U.S. provisional application Ser. No. 62/518,050 filed Jun. 12, 2017, the disclosure of which is hereby incorporated in its entirety by reference herein.

### TECHNICAL FIELD

In at least one aspect, the present invention relates to the treatment of diseases related to cell metabolic dysfunctions. The inventive solution relates to the fields of oncology, infectious disease and medicine. More particularly, the present invention provides pharmaceutical formulations of metal-anion complexes such as metal thiocyanate, salicylate, iodide and the like for the treatment of cancer and infections.

### BACKGROUND

Cancer is a metabolic disease that is resistant to many treatments, specifically those that target gene mutations. This is due, in part, by that fact that these gene mutations are remarkably diverse; such that a single tumor can have several different mutations within it. The diverse signaling pathways give rise to adaptive mutations, enabling cells to survive and multiply as they become resistant to new treatments. Furthermore, there has been a shift from characterizing cancer as metabolic disease to classifying it as an epigenetic response to chronic stress.

Since cancer cells typically have abnormal energy metabolism, the inventive treatment targets the most general characteristics of cancer, and other abnormal proliferative cells, that are distinct from normal cells.

Reduction-Oxidation reactions are essential to energy transduction in living organisms. This transduction occurs when electrons and protons flow through a liquid crystalline matrix into and out of the cell. Reduction occurs when the chemical species receives an electron, while oxidation occurs when the species donates an electron. This electric potential is essential in regulating the cells vitals states such as cell division, regeneration and proliferation (i.e. cancer). Cell potentials vary throughout the cycle, where high potentials correlate to less cell division and low potentials allow for malignant transformation. Cancer cells have an imbalanced redox potential, where the mitochondria are hyperpolarized. Thus, the need for an inventive complex that can restore homeostasis within cancer cells is needed.

The relationship between cancer and bio-metals such as iron, copper, zinc and manganese has been recognized over the years. Of those, iron facilitates cell proliferation and growth. Iron also has the capacity to engage in redox cycling and free radical formation—meaning it can also contribute to tumor initiation. Iron pathways are disturbed in tumor cells, and this reprogramming may be a central aspect of the survival of cancer.

Iron also plays a role between host and parasite interactions. During an infection, there is a battle between the host and the invader for iron supply. The acquisition of iron by a pathogen is a crucial step in infection, thus, the depletion of iron is one strategy to combat diseases.

Zinc is also essential for cellular functions. Depleted levels of zinc can affect cell functions and subsequently lead to a frequency of infections. Furthermore, patients with this depletion have shown increased levels of reactive oxygen species (ROS) and pro-inflammatory cytokines. Zinc is necessary for the function of over 300 enzymes and inhibits transcription factors, kinases and phosphatases; it also assembles multi-protein complexes. Zinc also regulates gene expression, where these genes play a role in signal transduction by regulating immune response to oxidative stress or energy consumption.

Non-protein bound, intracellular zinc plays a considerable role in regulation cellular functions as it relates to cancer. For example, increased free zinc has been proposed to stabilize hypoxia-inducible factor-1 (HIF-1), thus influencing glycolysis, apoptosis and angiogenesis. Furthermore, zinc inhibits thioredoxin reductase—a key mediator in the cells response to increased oxidative stress.

Zinc is a co-factor to many enzymes involved in epigenetic regulation. These enzymes include DNA methyltransferases, methyl binding proteins and histone-modifying enzymes such as acetylates, deacetylases or methylases. Furthermore, zinc deficiency induces DNA hypermethylation. These facts point to zinc's essential role in epigenetic processes such as chromatin remodeling, DNA methylation, histone modification and noncoding RNA syntheses.

The zinc dependent enzyme, carbonic anhydrase, is responsible for maintaining the acid/base equilibrium in the body. Acid/base equilibrium is the process by which the body keeps its pH as neutral as possible (at pH=7). Carbonic anhydrase helps convert carbon dioxide and water into bicarbonate and water to achieve this balance. Zinc depletion has shown reduction in red blood cell carbonic anhydrase activity.

Zinc is highly concentrated in the cerebral cortex, penal gland and hippocampus. In the hippocampus, zinc concentrations can reach up to 8%. When these levels are depleted, cognitive functions are affected, resulting in impaired memory function and mood disorders.

Some zinc compounds have been proposed for cancer treatment, including zinc chloride for skin cancer. However, its corrosive nature hampered its medical application. Thus, the need for a useful zinc containing compound for cancer treatment is essential.

There is a rapid decrease of free zinc in response to bacterial endotoxins and cytokines. For instance, zinc regulates the innate immune response to polymicrobial sepsis via NF-kB. In addition, the cytosolic and lysosomal availability of zinc in phagocytic cells is regulated in response to infections by reducing the availability of zinc to bacteria.

Salicylic acid is a plant hormone that is chemically like the active ingredients in aspirin. There is plenty of data showing that when aspirin is taken regularly it decreases the risk of colon cancer development by an average of 50% as compared to nonusers. Salicylate metal complexes have higher bioavailability and show less side effects than its aspirin counterpart and may have similar cancer related effects.

Several metal salts have been developed for cancer therapy, such as colloidal lead phosphate and a variation of copper, nickel and cobalt butylphthalate complexes.

Thiocyanate salt, in potassium form, was proposed as a method to treat hypertension. This treatment inhibited normal preneoplastic and neoplastic mammary gland development in mice that had decreased T3 and T4 plasma levels. However, the secretion of pituitary and ovarian mammotropic hormones was unaffected.

Though there have been many promising developments in the development of cancer treatments, most have failed due to their toxicity and lack of successful trials.

Accordingly, there is a need for improved compositions and methods for treating cancer, chronic obstructive pulmonary diseases, or inflammatory diseases.

### SUMMARY

The present invention solves one or more problems of the prior art by providing a method for treating cancer, chronic obstructive pulmonary disease, asthma, diabetes, sickle cell disease, thalassemia, or any other inflammatory disease, and/or promoting healing thereafter. The method includes a step of identifying a subject afflicted with cancer, chronic obstructive pulmonary disease, or an inflammatory disease. A therapeutically effective dose of a pharmaceutical composition is administered to the patient. The pharmaceutical composition includes at least one metal-containing compound. Characteristically, the at least one metal-containing compound includes a metal selected from the group consisting of zinc, copper, manganese, and combinations thereof.

In one aspect, a treatment using a zinc complex, including one zinc ion and a suitable anion such as salicylate, thiosulfate, iodide or the like, is proposed for the treatment of cancer, pulmonary diseases, inflammation related diseases and other diseases related to defective metabolic functions. Instead of targeting these diseases by repairing genetic mutations and destructive cells, this method will optimize mineral transport of compounds into the cell and correct cellular oxidation-phosphorylation. As a result, the cells will be better equipped to inhibit cancer growth and reduce the damaging effects of tumors to body tissue. These inventive combinations also effectively treat advanced cancer symptoms like pain, cachexia and hypercalcemia.

In another aspect, the treatment methods eliminate or retard tumor growth, prevent metastasis, improve symptoms, eliminate pain and prolong survival of the host.

In another aspect, the treatment methods can further be used in relation to bacterial and fungal infections, parasitic infections, skin psoriasis and neurogenerative disorders.

In another aspect, a method for treating cancer, chronic obstructive pulmonary disease, asthma, diabetes, sickle cell disease, thalassemia, or any other inflammatory disease, and/or promoting healing thereafter is provided. The first step is identifying a subject afflicted with cancer, chronic obstructive pulmonary disease, or an inflammatory disease. The method then consists of administering a therapeutically effective dose of a pharmaceutical composition comprising a zinc-containing molecule and a sulfur-containing anion.

In another aspect, a method for treating, ameliorating or inhibiting cancer, cancer related disorders or inflammatory diseases is provided. The method includes a step of identifying a subject having cancer, cancer related disorders and/or inflammatory disease. The method then consists of administering a therapeutically effective amount of at least one salicylate-metal complex or any combination thereof or any pharmaceutical composition comprising a salicylate-metal complex.

In another aspect, a pharmaceutical composition including a zinc-containing molecule and a sulfur-containing anion is provided.

In another aspect, a pharmaceutical composition includes a metal salicylate selected from the group consisting of zinc salicylate, copper salicylate, manganese salicylate and combinations thereof, and a pharmaceutical carrier.

In another refinement, the metal ions used, in combination with the aforementioned anionic complexes, can be manganese, copper or the like.

Therefore, this new embodiment proposes new combinations of the compounds introduced above, such as zinc thiocyanate and zinc salicylate, or suitable pharmaceutical derivatives thereof. These complexes are more effective and less toxic treatments for cancer and other diseases such as COPD, diabetes, sickle cell diseases, inflammation related diseases and many others.

These pharmaceutical compositions, in effectively permeating the cell, inhibit cancer cell function by depolarizing mitochondrial membrane potentials. In addition, these complexes provide materials to restore reduction/oxidation equilibrium back to cancer cells.

The treatment provides effective ways to transport zinc- and transition metal-containing complexes into cells effectively, to combat tumor growth. In addition, this treatment has strong clinical benefits for other diseases including chronic obstructive pulmonary disease (COPD), asthma, diabetes, sickle cell diseases, thalassemia and other inflammatory related diseases. These treatments can be administered orally, systematically, parenterally, topically, or through medicinal electrophoresis.

In another aspect, the methods herein use one of two ways to increase metal (e.g., Zn) uptake into cells—complex with selected anion and the use of nanoparticles. Therefore, nanoparticles can be administered to a patient using a metal salt (e.g., zinc salt) set forth herein as the carrier into the body.

In another aspect, zinc ionophores and zinc chelators, which have enhanced aqueous solubility, are used to transport molecules in the treatment of cancer. These complexes are analogues of pyrithone and have the potential to bind to zinc and carry it into the cell. However, pyrithone carriers are extremely toxic to the cell.

### BRIEF DESCRIPTION OF THE DRAWINGS

FIGS. 1A-J are bar graphs showing Zn-Salicylate dependently inhibits cell metabolism and proliferation in various cancer cells using CCK and MMT assays.

FIG. 2 is a bar chart showing that Zn-SA inhibits HepG2 cell proliferation in 3D gel conditions.

FIGS. 3A-D are bar charts showing that Zn-SA inhibits various cancer cell lines in 3D cultures at 3% gel concentration.

FIGS. 4A-C are images showing the effect of treatment versus non-treatment of HepG2 with Zn-SA on cluster formation

FIG. 5 is a schematic of a 3D co-culture model is used to culture liver cancer cells and fat cells.

FIGS. 6A-I provides experiment results demonstrating Zn-SA inhibits cancer cells proliferation, their associated ATP levels and cell count, but not normal cells.

FIG. 7 provides experimental results demonstrating the quick action of Zn-SA on cancer cells, indicating the high bioavailability of Zn-SA that transported into the membrane.

FIG. 8 shows MTT assays showing that Zn-SA is necessary for the biological function, and that salicylic acid alone has no effect on cellular activity.

FIGS. 9A and 9B are bar charts showing that Zn-SA is necessary for biological function and salicylic acid alone has no effect on cellular activity.

FIG. 10 provides microscopic images showing Zn-SA treated cells were efficiently cleared by microphage without inflammation.

FIG. 11 provides microscopic images showing Zn-SA treated cells were efficiently cleared by microphage without inflammation.

FIG. 12 provides microscopic images showing Zn-SA treated cells were efficiently cleared by microphage without inflammation.

FIG. 13 provides microscopic images showing Zn-SA treated cells were efficiently cleared by microphage without inflammation.

FIG. 14 provides microscopic images showing Zn-SA treated cells were efficiently cleared by microphage without inflammation.

FIG. 15 provides microscopic images showing Zn-SA treated cells were efficiently cleared by microphage without inflammation.

FIG. 16 shows the reduction of tumor size in treated mice.

FIG. 17 is an image showing control tumor (Left) and tumor necrosis (Right) after mice were treated.

FIG. 18 is a bar graph showing the relative staining density of Zn formed with four different anions. The complex shows that ZnSCN has the greatest relative density and lowest free Zn.

FIG. 19 shows four stained cell complexes viewed under a fluorescent microscope. The complex with greatest relative density is ZnSCN.

FIG. 20 is a bar graph showing the viability of fibroblast cell and three different liver cancer cells after being treated with ZnSCN over a 150-min period. The integrity of the ZnSCN treated cells decreased more than the integrity of the fibroblast cells.

FIGS. 21A and 21B shows two bar graphs that compare the viability of normal adipose stromal cells and ASPC-1 cancer cells when treated with eight different metal complexes. Zn, Mn and copper alone, or in combination, inhibit tumor cell viability but not normal stromal cell viability.

FIG. 22 shows images of mitochondrial membrane potentials of cells in a control group and those treated with ZnSCN under a fluorescence microscope. This shows that ZnSCN is effective in treating cancer by de-polarizing the mitochondrial membrane potential.

FIG. 23 comprises of two images that compare the presence of acidic vascular organelles in cells of a control group to those treated with ZnSCN.

FIG. 24 is a graph comparing tumor sizes in animals treated with ZnSCN and an untreated control group.

FIG. 25 is a chart comparing the anti-tumor effect of metal complex treatments. A group was left untreated, another was given ZnSCN+Mn orally and another was treated with ZnSCN+MnSA+CuSA subcutaneously.

FIG. 26 is a plot showing that ZNS nanoparticle dose dependently inhibits breast cancer cell MDA-MB-231 cell proliferation.

FIG. 27 are micrographs showing that ZnS slows cell proliferation but doesn't induce cell death compared to chemo drug Taxol.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Throughout this application, where publications are referenced, the disclosures of these publications in their entireties are hereby incorporated by reference into this application to more fully describe the state of the art to which this invention pertains.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms are used, the presently disclosed and claimed subject matter can include the use of either of the other two terms.

The term “subject” refers to a human or animal, including all mammals such as primates (particularly higher primates), sheep, dog, rodents (e.g., mouse or rat), guinea pig, goat, pig, cat, rabbit, and cow.

Abbreviation:

“SA” means salicylate.

“MT” means metallothionein.

In an embodiment, a method for treating cancer, chronic obstructive pulmonary disease, asthma, diabetes, sickle cell disease, thalassemia, or any other inflammatory disease, and/or promoting healing is provided. The method includes a step of identifying a subject afflicted with cancer, chronic obstructive pulmonary disease, or an inflammatory disease. A therapeutically effective dose of a pharmaceutical composition is administered to the patient. The pharmaceutical composition includes at least one metal-containing compound. In a refinement, the at least one metal-containing compound includes a metal selected from the group consisting of zinc, copper, manganese, and combinations thereof. The at least one metal-containing compound can be in the form of an organic or inorganic salt, complex, chelator, nanoparticle, micron-sized particle or ionophore. In a preferred application, the pharmaceutical composition is effective at slowing primary or metastatic tumor growth and causing regression of cancer in an afflicted subject.

The at least one metal-containing compound can be described by MXₙ wherein M is a metal ion (e.g., zinc, copper, manganese ions), X is a counter ion, n is the number of X to maintain charge neutrality (e.g., 1, 2, or 3). Examples of X include sulfate, O (e.g., to form ZnO), S (e.g., to form ZnS), halide (e.g., chloride, bromide, iodide), gluconate, ascorbate, lactate, laurate, peroxide, salicylate, thiocyanate, aspartate, benzoate, and the like. In a particularly useful variation, the metal-containing compound is a metal halide, and in particular zinc iodide.

In one variation, the pharmaceutical composition includes a sulfur-containing anion. In this regard, the metal-containing compound can include the sulfur-containing anion. In a refinement, the pharmaceutical composition further comprises a second compound that includes the sulfur-containing anion. Examples of suitable sulfur-containing anions include, but are not limited to thiocyanate, isothiocyanate, thiosulfate, methionine, and combinations thereof. A particularly used compound that includes a sulfur-containing ion is zinc thiocyanate which is effective at inhibiting cancer cell function by depolarizing mitochondrial membrane potentials. In a refinement, the pharmaceutical compositions that include sulfur-containing anion can include a second metal-containing compound such as of zinc salicylate, copper salicylate, manganese salicylate, and combinations where the first metal-containing compound is different than the second metal containing compound.

In a refinement of the variation that includes the sulfur-containing anion, the pharmaceutical composition includes one or more addition metal-containing compounds having formula MXₙ as set forth above. For example, the pharmaceutical composition can include at least one compound selected from zinc salicylate, copper salicylate, manganese salicylate, zinc gluconate, copper gluconate, manganese gluconate, zinc aspirinate, copper aspirinate, and manganese aspirinate. A particularly useful combination includes zinc thiocyanate, manganese gluconate, and copper gluconate.

In a variation, the metal-containing compound is a metal salicylate complex selected from the group consisting of zinc salicylate, copper salicylate, manganese salicylate, and combinations thereof. In this variation, zinc salicylate is found to be particularly useful. For this variation, a dose of the metal salicylate complex is 10 to 1000 mg of metal salicylate/50 kg of body weight per day is found to be useful. In a refinement, a dose of the metal salicylate complex is from 0.04 to 10 mg of metal salicylate/kg of patient body weight per day. In a refinement, the pharmaceutical composition of this variation further includes succinic acid. A particularly useful pharmaceutical composition of this variation includes zinc salicylate, copper salicylate, manganese salicylate, and succinic acid.

In a refinement of the salicylate-containing compositions, the pharmaceutical composition includes a combination of a therapeutically effective amount of at least two salicylate-metal complexes. In a refinement, the composition optionally further comprises at least one pharmaceutically acceptable carrier, diluent, excipient and/or additive. Specific embodiments of the invention relate to combined compositions comprising a combination of a zinc salicylate complex with a copper salicylate complex. In another variation, the pharmaceutical composition includes zinc salicylate, copper salicylate, manganese salicylate and succinic acid. In a further refinement, the pharmaceutical composition further includes another compound of formula MXₙ as set forth above. In particular, this additional metal-containing compound is a gluconate.

In another variation, the metal-containing compound is an aspirinate-metal complex selected from the group consisting of zinc aspirinate, copper aspirinate, manganese aspirinate, and combinations thereof.

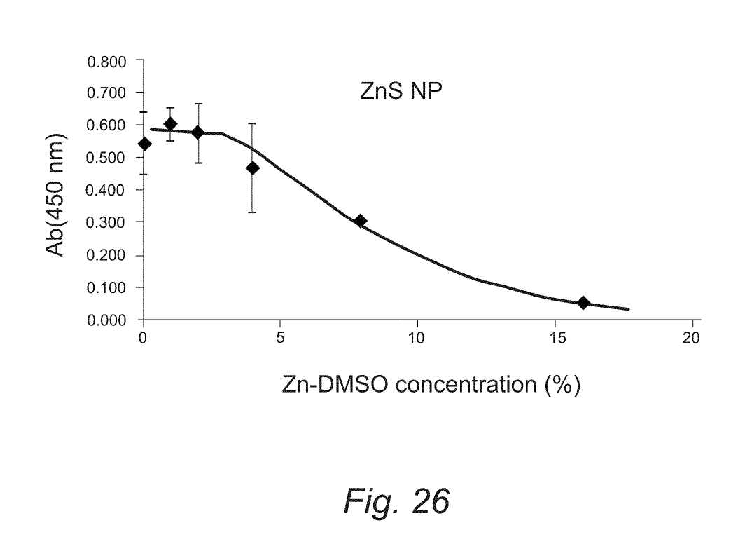

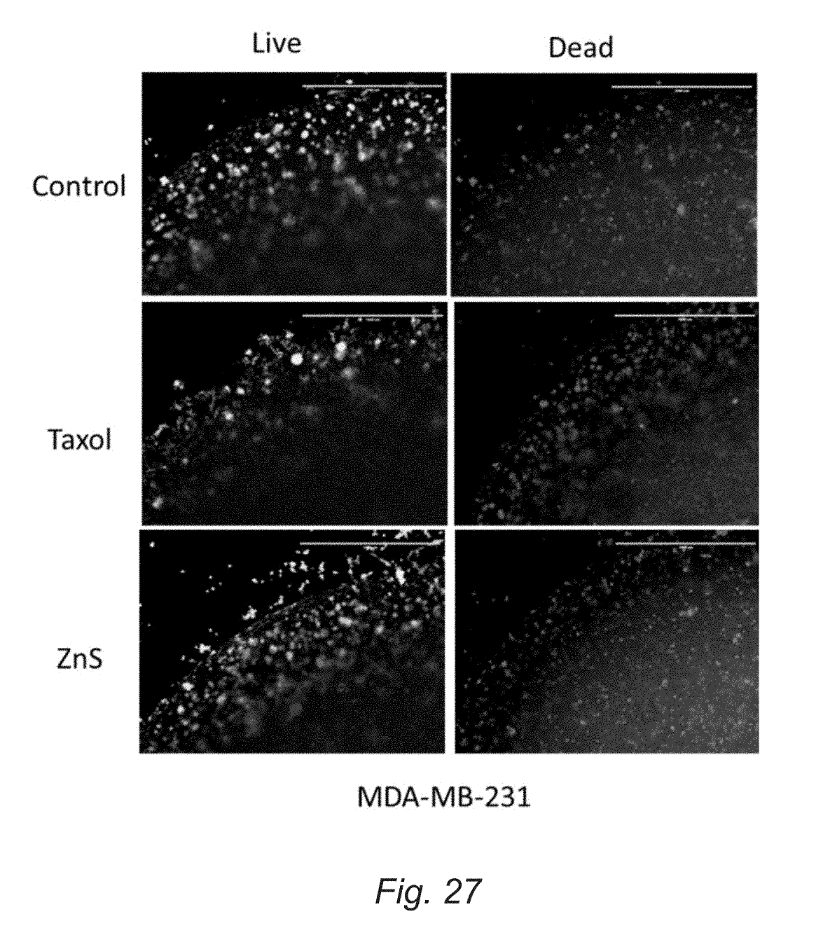

In another variation, nanoparticles of the metal-containing compounds set forth herein are found to be particularly useful. Typically, the nanoparticles have an average size (i.e., average diameter) from about 20 to 100 nm. In this regard, zinc sulfide is found to be particularly useful. In this regard, different pH values in the environment will result in different forms of sulfide (S2-, HS- or H2S), thus controlling pH plays an important role in ZnS production in the biosynthesis. In a refinement, the nanoparticles are capped with DMSO, thiocyanate, salicylate, PEG, tannin, polyflavonoids, Tannin, etc to prevent particle aggregation and increase water solubility. (See FIGS. 26 and 27.)

The pharmaceutical compositions set forth above can include one or more suitable pharmaceutical carriers, solvents buffers which are suitable for complex formation and/or required for prolonged shelf-life of the treatment. In another variation, the composition can be combined with DMSO and/or succinate for cellular delivery. In particular, the pharmaceutical compositions set forth above can include the pharmaceutical composition and can further include a fluid carrier biocompatible with human tissues. Such fluid carriers further may have physiological strength electrolytes dissolved therein for this purpose. In some refinements, the fluid carrier can also include additives that can be water soluble, water miscible, or aqueous suspensions. Typically, such additives are collectively present in an amount from about 0 to 20 weight percent of the total weight of the pharmaceutical composition. In a refinement, these additives are present in an amount from about 0.1 to about 15 weight percent of the total weight of the pharmaceutical composition and individually in an amount from about 0.05 to about 5 weight percent of the total weight of the pharmaceutical composition. Examples of such additives include, but are not limited to, minerals, medical solvents, ethanol, coloring agents, antimicrobial preservatives, blending agents, and combinations thereof. In additional refinements, the fluid carrier further can include a surfactant typically in a concentration of about 0.05 to 2%. In a refinement, the carrier fluid also contains one or more conventionally known surfactants in a concentration of about 0.05 to 2%. The surfactants could have anionic, cationic, nonionic, or ampholytic properties.

In some refinements, the pharmaceutical carriers include, but are not limited to gelatin capsules; sugars such as lactose and sucrose; starches such as corn starch and potato starch; cellulose derivates such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, corn oil, olive oil, and oil of theobroma; propylene glycol; glycerin; sorbitol; polyethylene glycol; water; agar; alginic acid; isotonic saline and phosphate buffer solutions; as well as other compatible substances normally used in pharmaceutical formulations. The compositions of the invention can also contain coloring agents, flavoring agents, and/or preservatives. These materials, if present, are usually used in relatively small amounts (e.g., each independently about 0.05 to 5 weight percent).

The pharmaceutical compositions set forth above can be administered by any number of methods known to those skilled in the art including orally, parenterally, transdermally, medical electrophoretically, and the like. For oral administration, the pharmaceutical compositions set forth above can be administered through tablets, capsules, powder or liquid form. The orally administered dose could be 60 to 200 mg over a 24-hour period. In this embodiment, the pharmaceutical composition could be produced in tablets, capsules, powder, or liquid form. In a variation, administration is done parenterally by infusion or injection. The dose could be 0.5 to 10 mg/kg subject body weight over a 24-hour period. In a preferred variation, the dose is 2 to 8 mg/kg subject body weight in 24 hours. In an optimal variation, the dose is between 4 and 6 mg/kg subject body weight, administered over a 24-hour period. In an alternate variation, administration of the pharmaceutical compound is done topically in a dose of 10 to 1000 mg. Another method of administration is medicinal electrophoresis, in which the dose would also be 10 to 1000 mg.

In an exemplified embodiment for oral administration, the treatment contains 300 mg of zinc thiocyanate, 50 mg of manganese gluconate and 15 mg of copper gluconate.

When the pharmaceutical composition is administered parenterally, the preferred fluid carrier is compatible with cells and body tissue. Thus, chemical additives and agents may be included in small quantities in addition to physiological strength electrolytes. These additives and agents can be water soluble, water miscible or aqueous suspensions. In addition, various minerals such as DMSO, ethanol, coloring agents, antimicrobial preservatives, bending agents and the like are present in the additives. The pharmaceutical composition for parenteral administrations will often include at least one surfactant. The surfactant may be anionic, cationic, ampholytic or nonionic. Preferred concentrations range between 0.05% to 2.0%.

As for parenteral administrations (e.g. intra-arterial, intravenous, intraperitoneal, intra-tumoral, etc. modes), effective concentrations of zinc thiocyanate ranges from 0.5 mg/kg to 10.0 mg/kg subject body weight over a 24-hour period. However, the optimal concentration ranges from 4.0 mg/kg to 6.0 mg/kg subject body over a 24-hour period.

In an exemplified embodiment (e.g., oral administration), a dose of the pharmaceutical composition contains 20-200 mg of zinc thiocyanate, 5-25 mg of manganese gluconate, and 3-15 mg of copper gluconate. In an exemplified embodiment for oral administration, the treatment contains 300 mg of zinc thiocyanate, 50 mg of manganese gluconate and 15 mg of copper gluconate.

In an exemplified embodiment (e.g., parenteral administration), a dose of the pharmaceutical composition contains 200-600 mg of ZnSCN, 10 mL of 99.8% DMSO and 500 mL of 0.9% NaCl.

In a variation, suitable doses for the metal salicylate compositions set forth above are 10-100 mg of metal salicylate/50 kg and 0.2-20 mg of metal salicylate/kg/day for intravenous administration. In a refinement, the dose of salicylate with respect to Zn is 0.04 mg-4 mg/kg/day. For oral doses, a suitable range of metal salicylate 0.04-10 mg of metal salicylate/kg/day.

In a refinement, the pharmaceutical compositions set forth above that include sulfur-containing anion are administered in a therapeutically effective amount. In one refinement, the pharmaceutical composition is administered orally in an amount from 60 to 200 mg over a 24-hour period. In another refinement, the pharmaceutical composition is administered parenterally in an amount from 0.5 to 10 mg/kg subject body weight over a 24-hour period. In other refinements, the pharmaceutical composition is administered topically or by medicinal electrophoresis in an amount from 10 to 1000 mg administered topically or by medicinal electrophoresis.

In a refinement, the dose for the pharmaceutical composition includes a combination of a therapeutically effective amounts of at least two salicylate-metal complexes is in increasing order of preference 300 mg/50 kg/day (+/−40%), 100 mg/50 kg/day (+/−40%), 50 mg/50 kg/day (+/−40%) or 500 mg/50 kg/day (+/−40%) respectively. In particular, the dose for this variation is in a range from 300 to 700 mg/50 kg/day, 180 to 420 mg/50 kg/day or 30 to 70 mg/50 kg/day, in increasing order of preference.

For topical and electrophoresis administration, effective dosages range from 10-1000 mg with the proper carrier. In an exemplified variation for topical and transdermal use, the solvent used constitutes 80% DMSO and 20% water

The following examples illustrate the various embodiments of the present invention. Those skilled in the art will recognize many variations that are within the spirit of the present invention and scope of the claims.

Overview

In an embodiment, the Zn anion, or derivatives set forth above, inhibits cancer cell proliferation and metabolism. The net charge of the anion is positive and can pass through the membrane through passive diffusion. Normal cells, with proper ATP conjugates pumps, can deplete extra Zn out of the cell. Cancer does not have enough ATP to do this effectively. Thus, when the proper Zn anion (as proposed in the inventive solution) penetrates the cell and releases Zn into the cytoplasm, the cancer cell is killed.

The present invention does not solely depend on water solubility of zinc compounds, but also the net charge and molecule structure of the complex. ZnSCN, for instance, passes through the cytoplasm and mitochondrial membranes by passive diffusion, independent of any active ion transporters. The inventive solution does not pass through by one mechanism of action, nor does its success depend on its capability to dissolve in water to release the zinc. This is different than mechanisms stated in the prior art.

Superoxide dismutase (SOD) is an enzyme that breaks down superoxide radicals that are toxic to livings cells. SOD levels are diminished in tumors; and specifically, copper and zinc-containing SOD's are depleted. The reduction in SOD, and subsequent increase in radicals, contribute to tumor growth. Three different SODs have been found in eukaryotic cells; Cu/Zn type which binds to copper and zinc, Fe/Mn type which binds to iron and manganese, and Ni type which binds to nickel. SODs are in the cytoplasm, mitochondria and extracellular environment. SOD1 and SOD3 contain copper and zinc in their reactive center, while SOD2 contains manganese. In mammals, Cu/Zn types and Fe/Mn types are present. In all tumor activity, these enzymes are diminished; where the Fe/Mn type is undetectable. The depleted levels of the enzyme, along with continued production of superoxide radicals may contribute to the cancer phenotype. Thus, the reintroduction of these enzymes is vital for combating tumor growth.

Thiocyanate (SCN−) is a detoxification product of the reaction between cyanide and thiosulfates in the liver. The following are known to increase SCN− concentration in saliva and blood: foods containing cyanides (e.g. almonds, nuts, cabbage), medications (e.g. nitroprussine), and tobacco smoke.

Cellular metallothionein (MT) is one of the few eukaryotic proteins that control detoxification of heavy metals, such as zinc. The release of zinc from the MT-zinc complex is aided by sulfur containing proteins such as glutathione.

Transition metals are essential for many metabolic processes, so their constant homeostasis is essential for life. Deviations from cellular metal ion concentrations may lead to cell death and severe diseases. Metal ion transporters play a major role in maintaining correct concentrations of metal ions in cells. The results show that SCN− may be responsible for changes in these metals accumulation in the liver. SCN− inhibits the precipitation of proteins, such as MTs, leading to an alteration of cellular functional activities. Since MTs are essential to heavy metal detoxification, the presence of SCN− affects the presence of heavy metals, such as zinc in cells.

In mammals, SCN− is oxidized by peroxidases, such as myeloperoxidase and eosinophil peroxidase, into hypothiocyanite (OSCN−). This compound is innocuous to mammalian cells. When an oxidative burst occurs, oxygen free radicals and OSCN− are formed and likely contribute to leukocyte antimicrobial effect.

Results

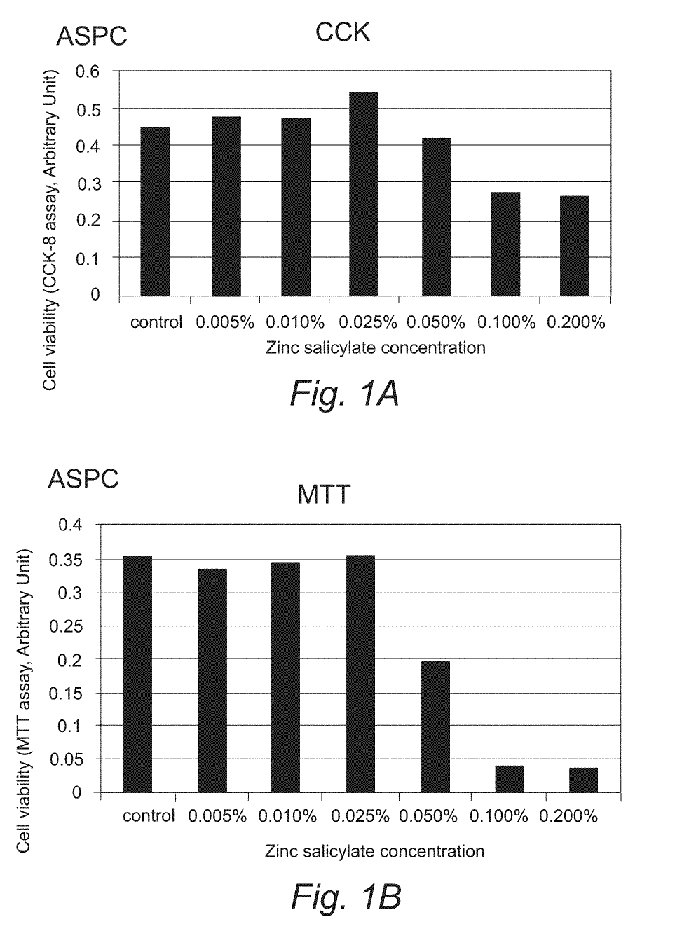

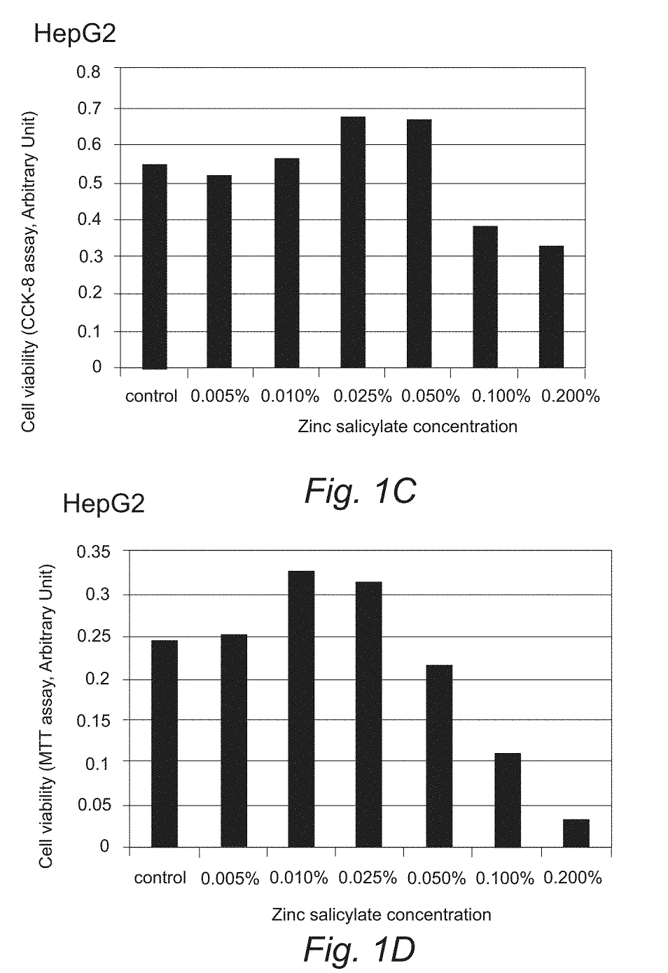

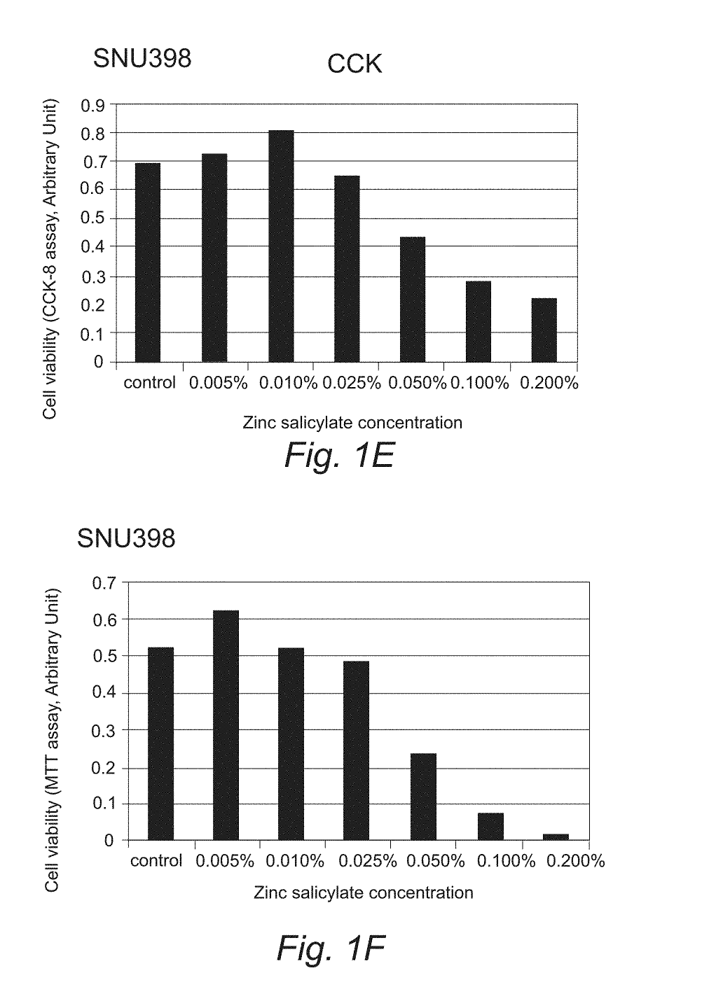

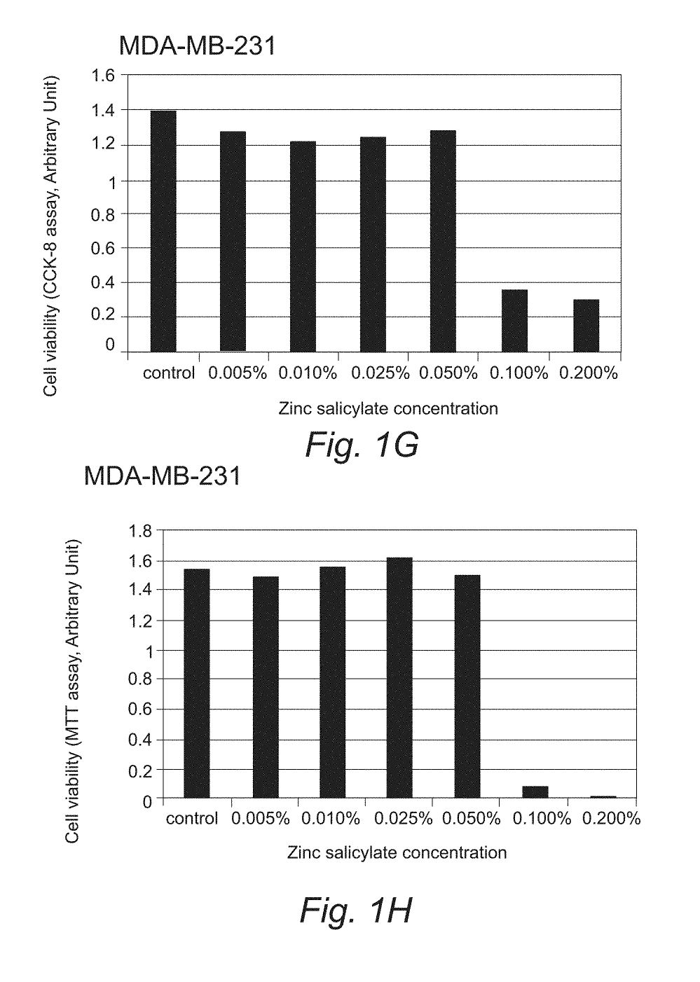

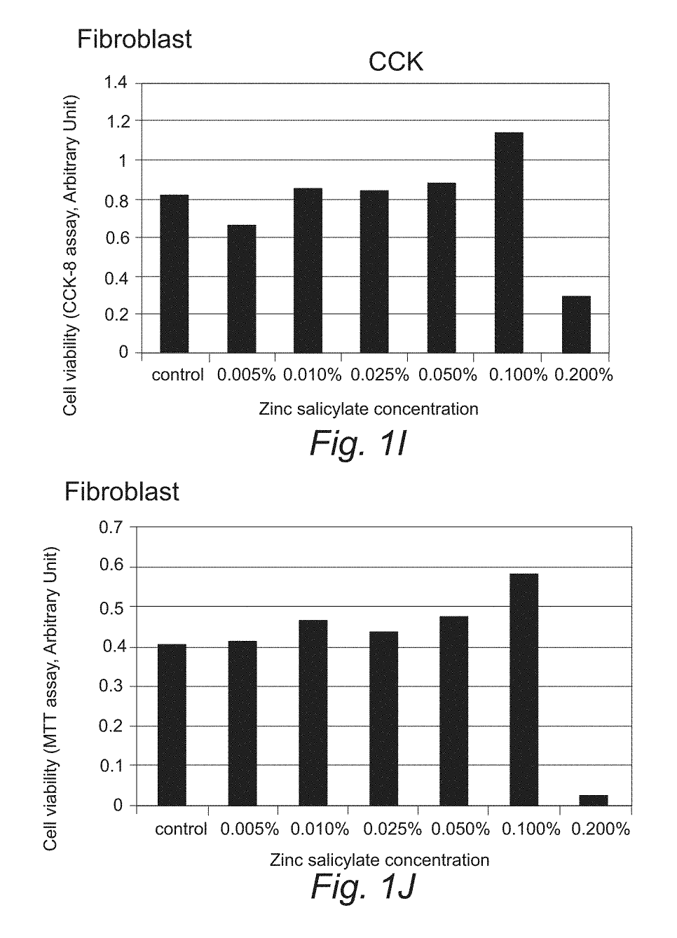

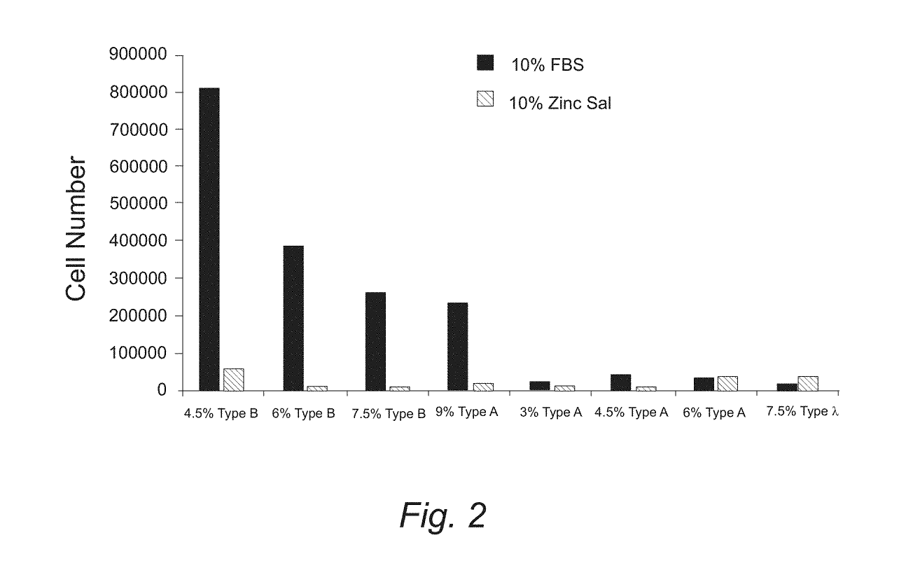

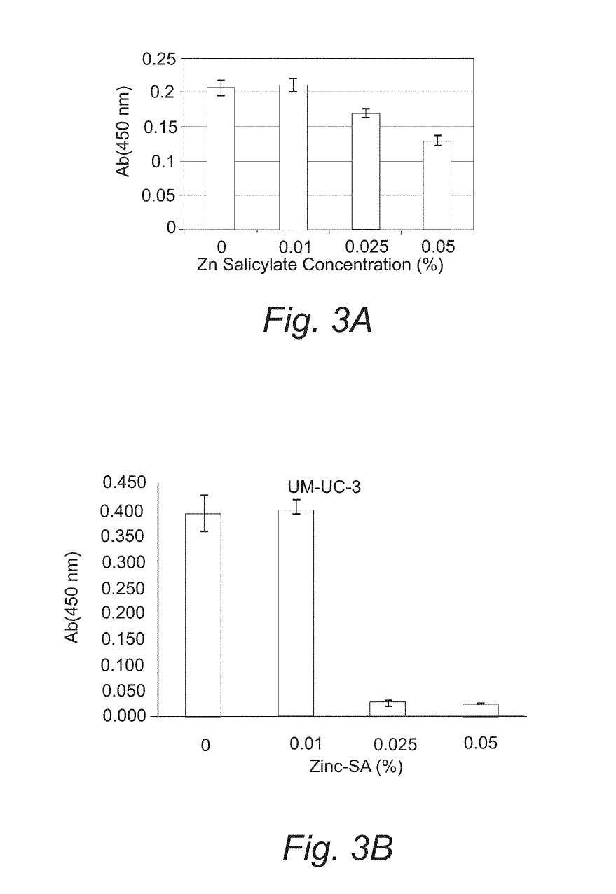

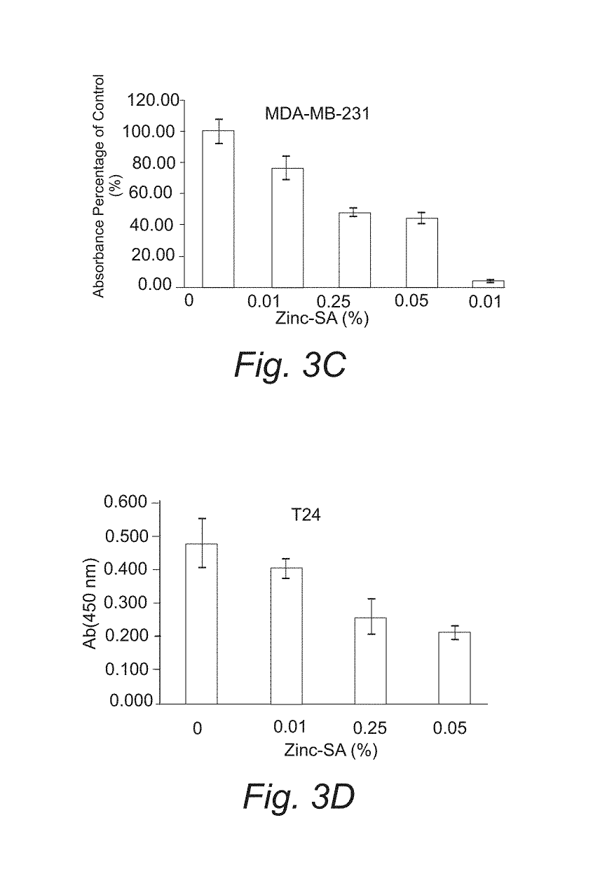

With reference to FIGS. 1A-J, an illustration showing that Zn-Salicylate dependently inhibits cell metabolism and proliferation in various cancer cells using CCK and MMT assays is depicted. Cell-lines were plated at 70% confluency and treated with Zn-SA 24 hours later. 48 hours after treatment, the cells were stained with MTT and dissolved for colorimetric reading, and then assayed by Cell-counting-Kit 8 (CCK-8) to indicate cell survival. Concentrations of 0.01%-0.1% Zn-SA inhibits cancer cell proliferation and survival rates. At the same concentration ranges, however, Zn-SA spares normal fibroblast, bone marrow and adipocyte cell proliferation.

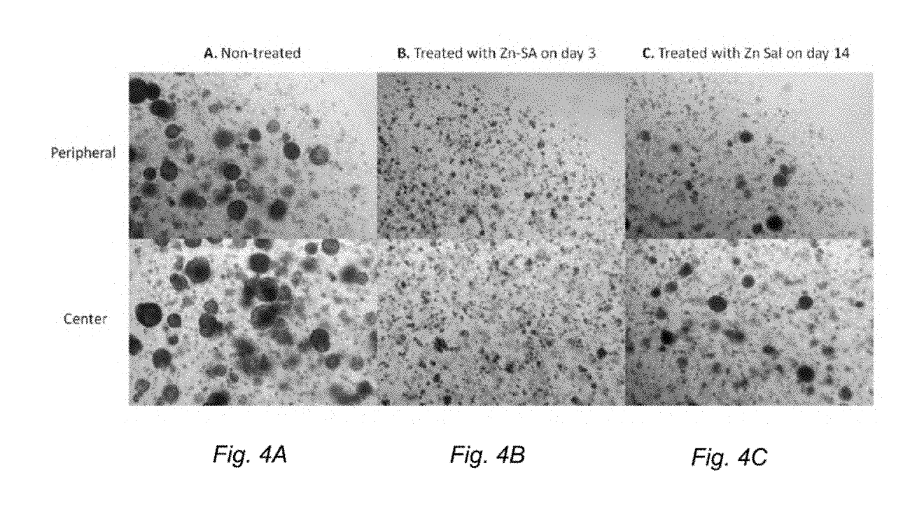

With reference to FIG. 4, the effect of Zn-SA on HepG2 cluster formation is illustrated. The cells that were left untreated all contained HepG2 clusters, as seen on day 17 of the 3D culture models. Another batch of cells were treated with Zn-SA on day 3 of gel formation. By the third week, no additional HepG2 clusters formed. In the third treatment, cells were injected with Zn-SA on day 14 of gel formation. The HegG2 levels were observed before and after treatment; and HeG2 clusters significantly diminished after Zn-SA treatment.

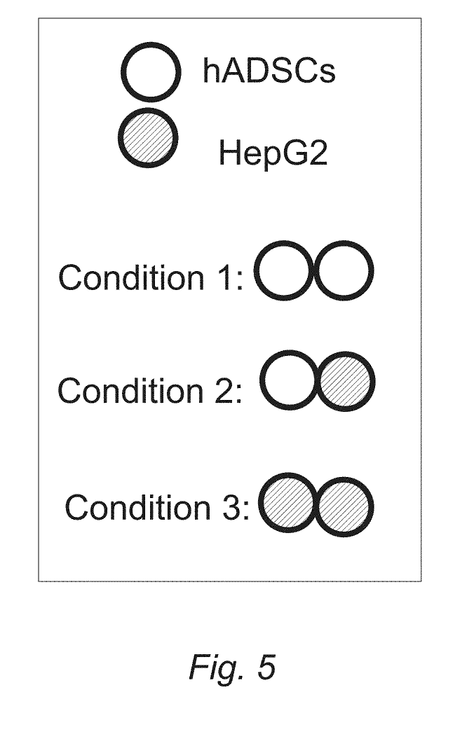

With reference to FIG. 5, a 3D co-culture model of liver cancer cells and fat cells is illustrated. Cells: human adipose derived stromal cells (hADSCs) and/or HepG2; culture medium: 10% FBS and 1% PS in MEM′ Gel conditions: 4.5%, 6%, 7.5%, and 9% type B gel and 3%, 4.5%, 6% and 7.5% Type A gel; Co-culture conditions: hADSCs+hADSCs, hADSCs+HepG2 and HepG2+HepG2; Treatment groups: control/non-treated; treated with 0.01% Zn-SA. On day 1, 10 microliters of each cell-gel construct were seeded together, as shown in the right. On day 3, the cells were treated with Zn-SA. On day 5, the medium was harvested for analysis. On day 6, the cells were harvested for cell count. The results are described below.

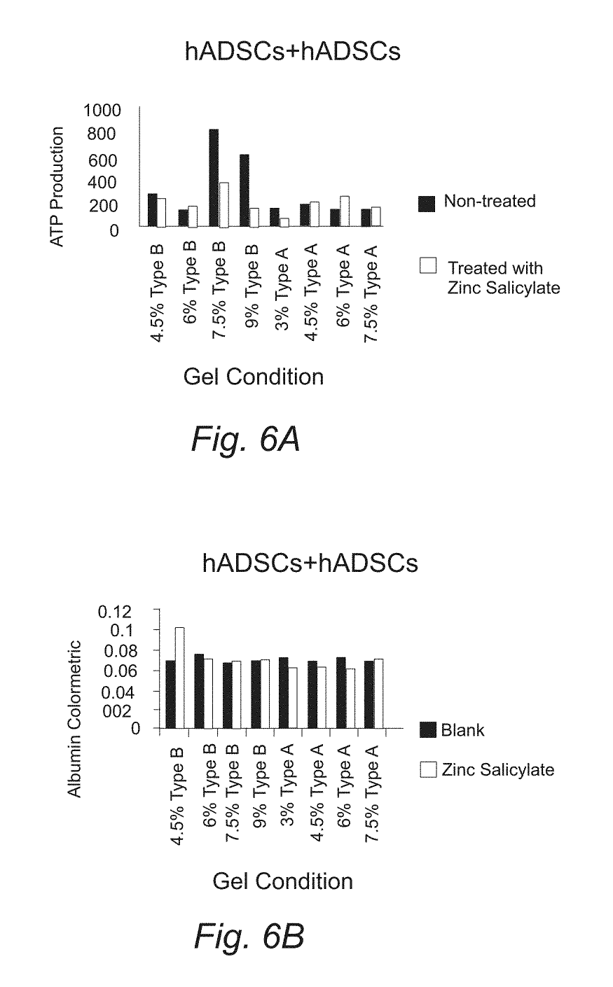

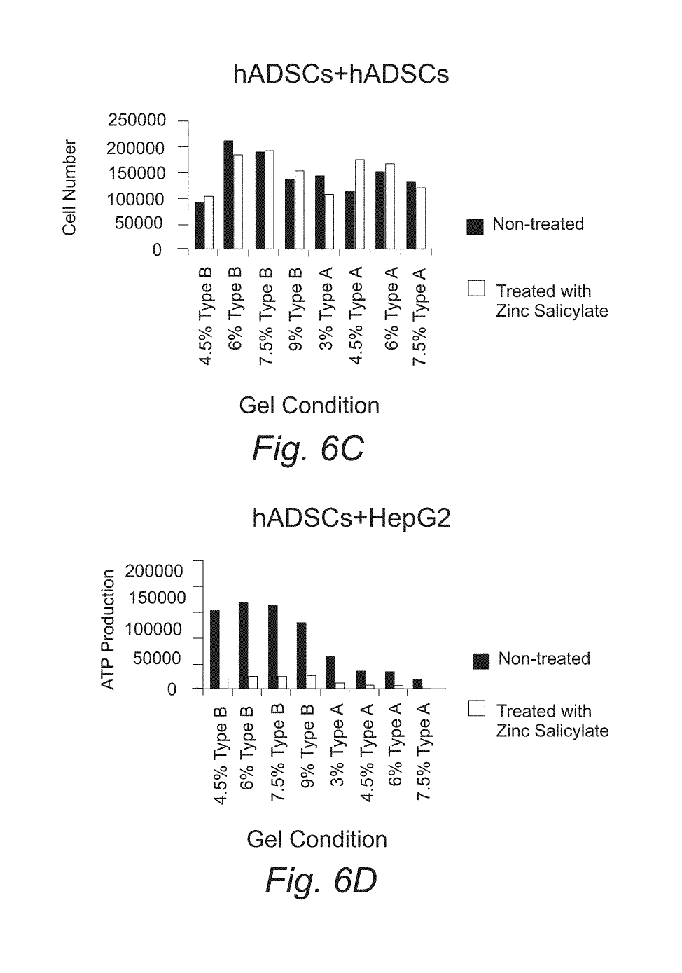

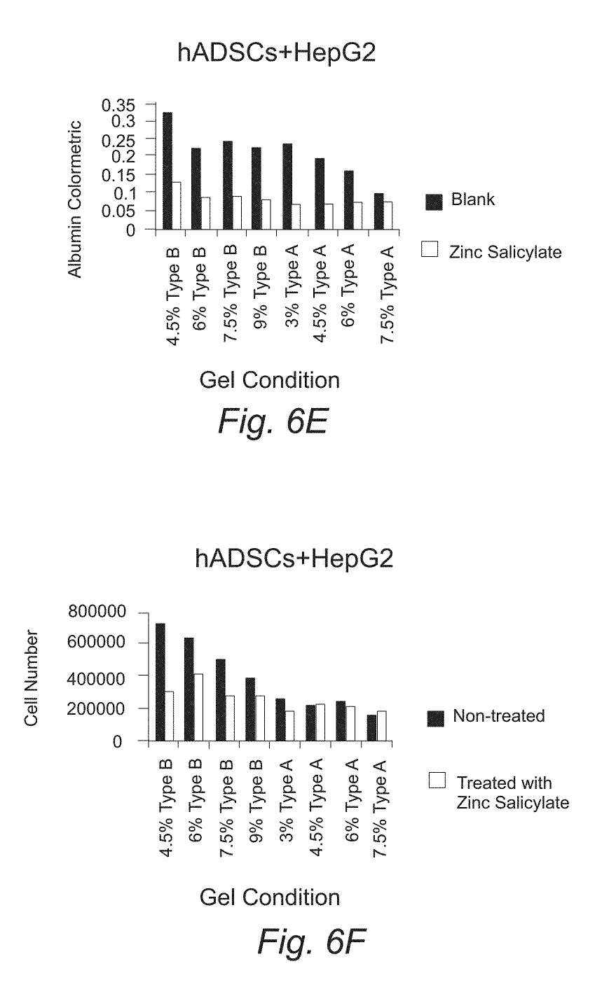

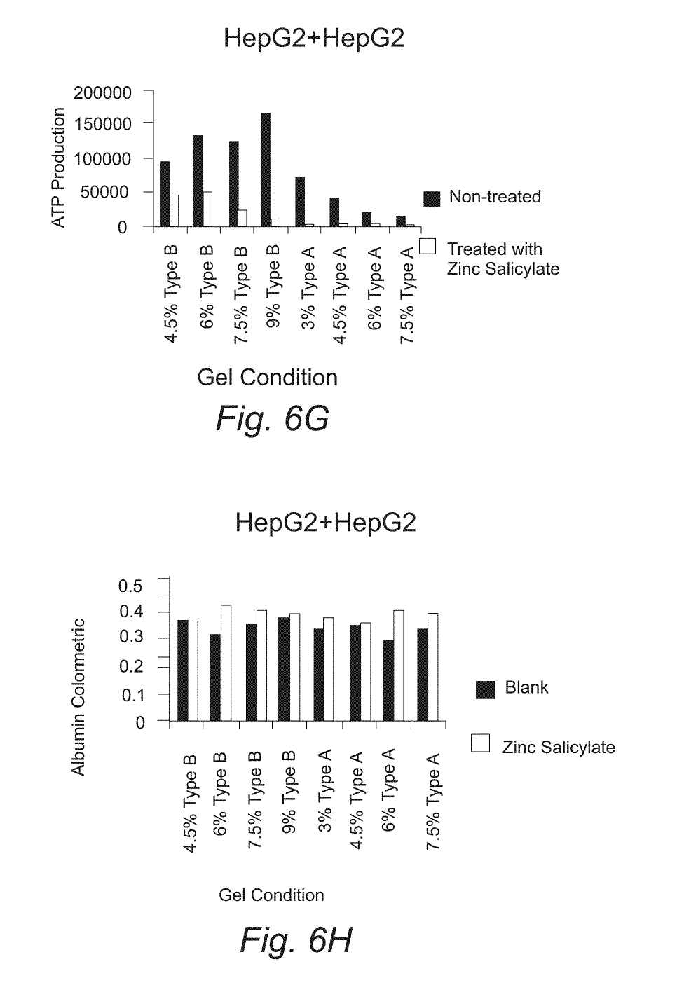

With reference now to FIG. 6, the effects of Zn-SA on hADSCs, HepG2 and the co-culture is illustrated. In hADSCs there were no significant changes in ATP levels, with an exception of the 7.5% and 9% Type B gels, albimun production or cell count. In HepG2, ATP production and cell count were significantly reduced, while albimun production remained unchanged. In the co-culture, ATP production, albumin production and cell count significantly decreased. The results show that hADSCs aid cell proliferation, ATP and albumin production of HepG2.

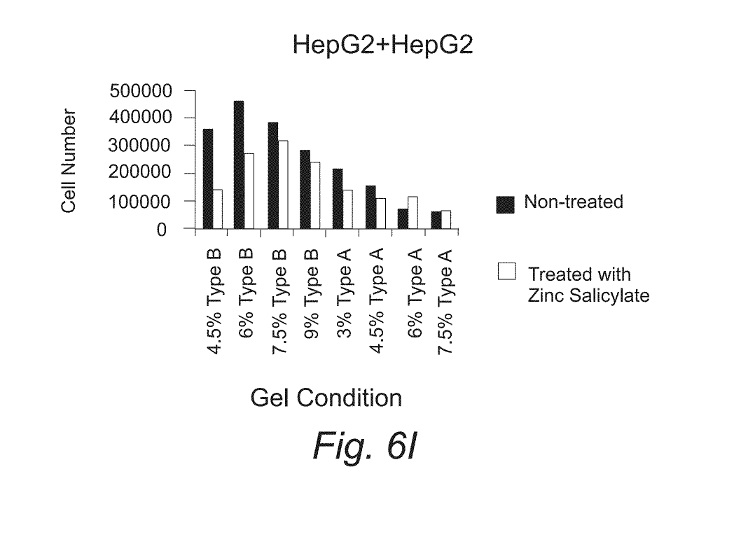

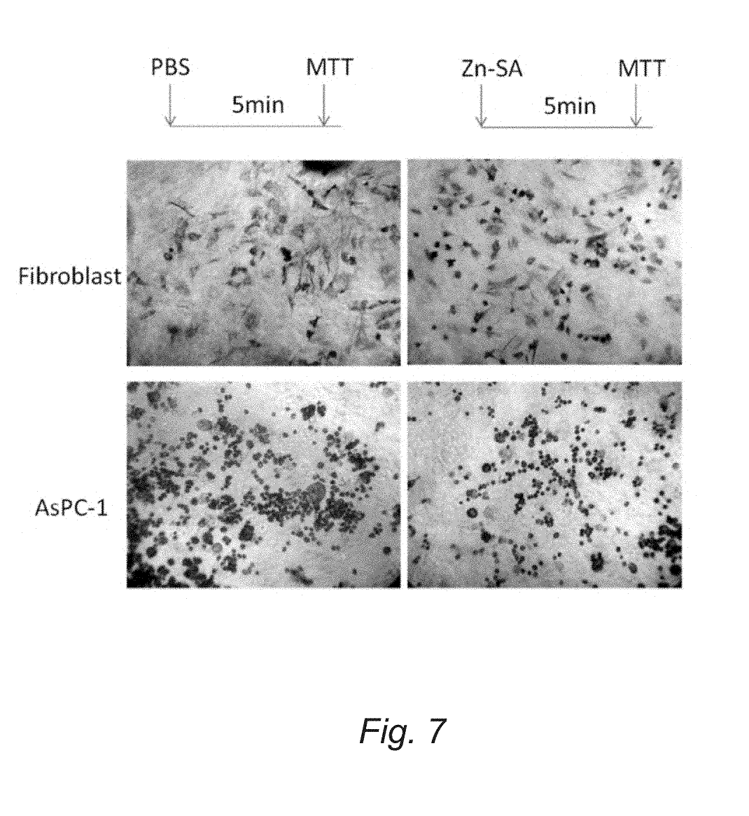

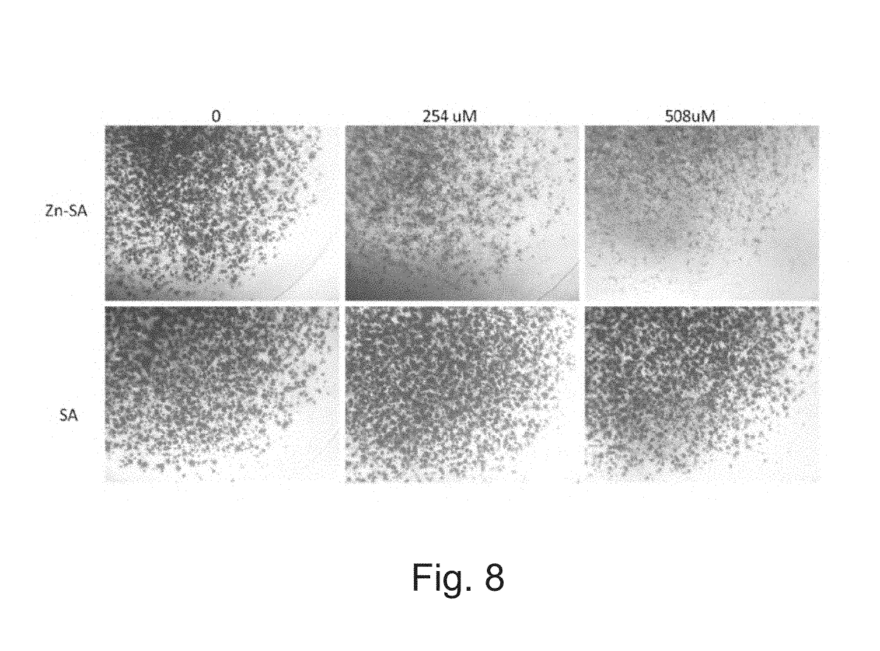

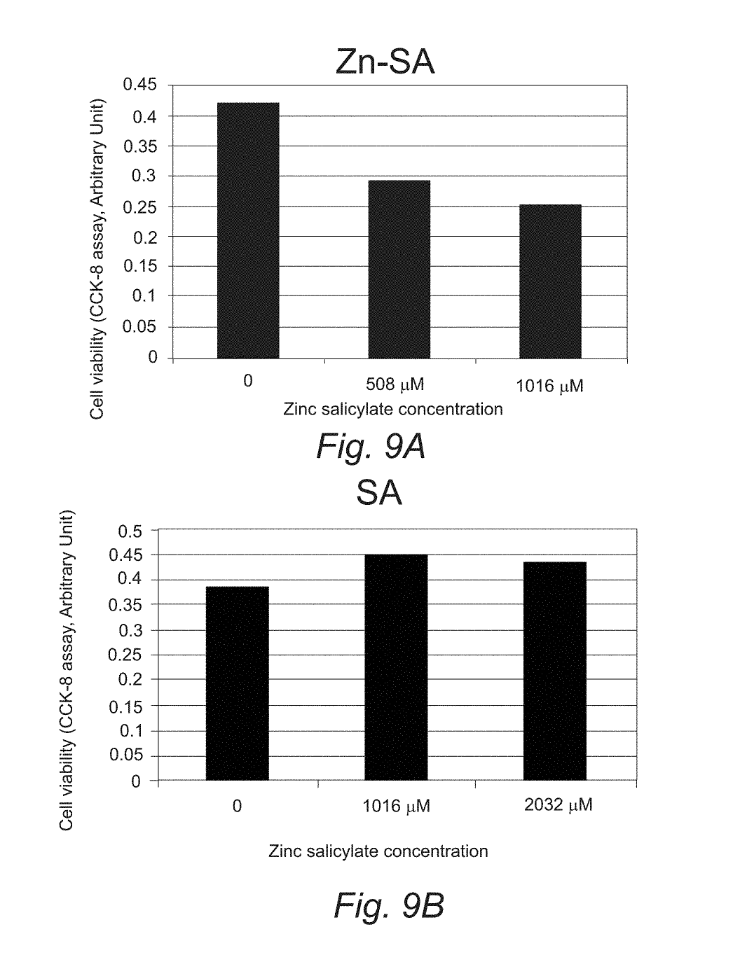

With reference now to FIG. 7, the ability of Zn-SA to penetrate cells and inhibit hydrogenase function of tumor cells is illustrated. Normal fibroblast and pancreatic cancer AsPC cells were cultured in a 3D gel. Zn-SA (0.05%) was added in the culture well five minutes before MTT dye was added. In the normal cells, the uptake rate of MTT in Zn-SA treated and non-treated cells was similar. However, in the AsPC cells, the MTT uptake was significantly less in the Zn-SA treated group as opposed to the non-treated group.

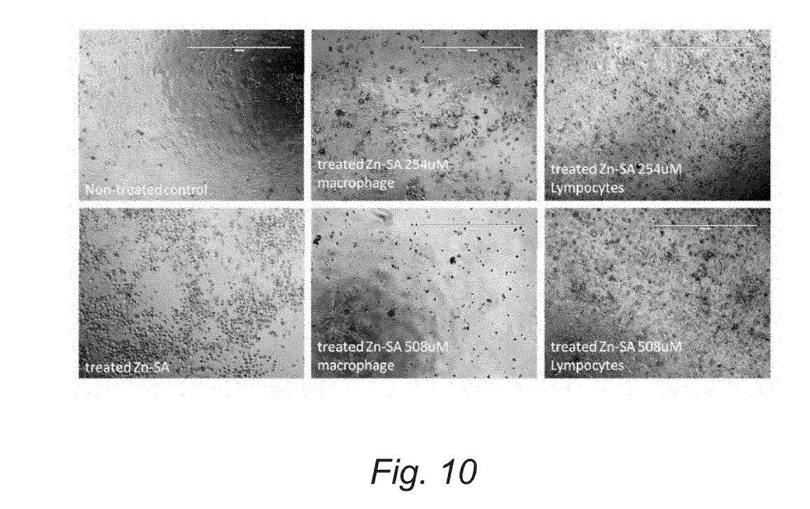

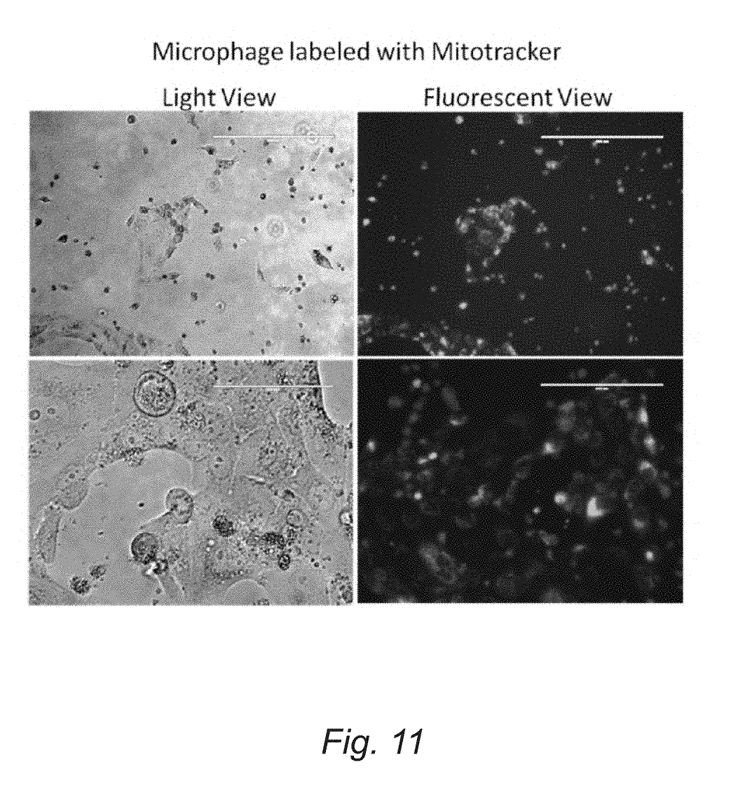

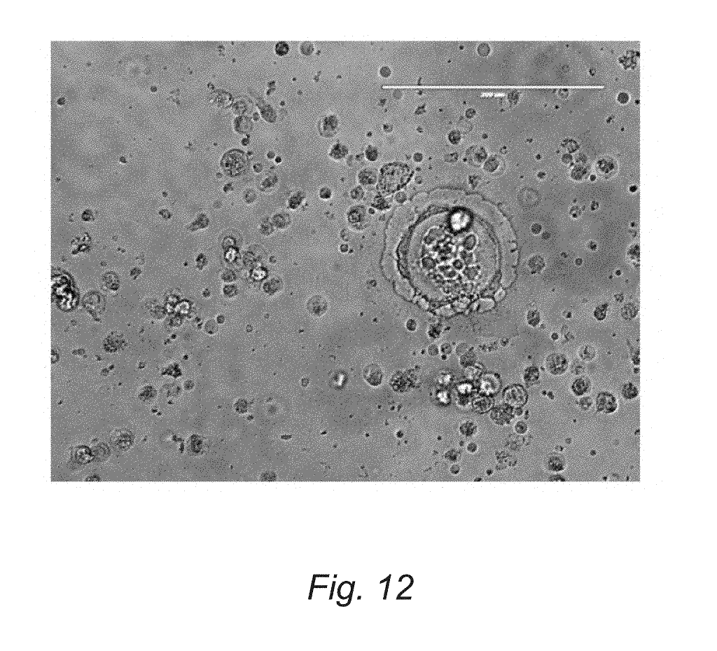

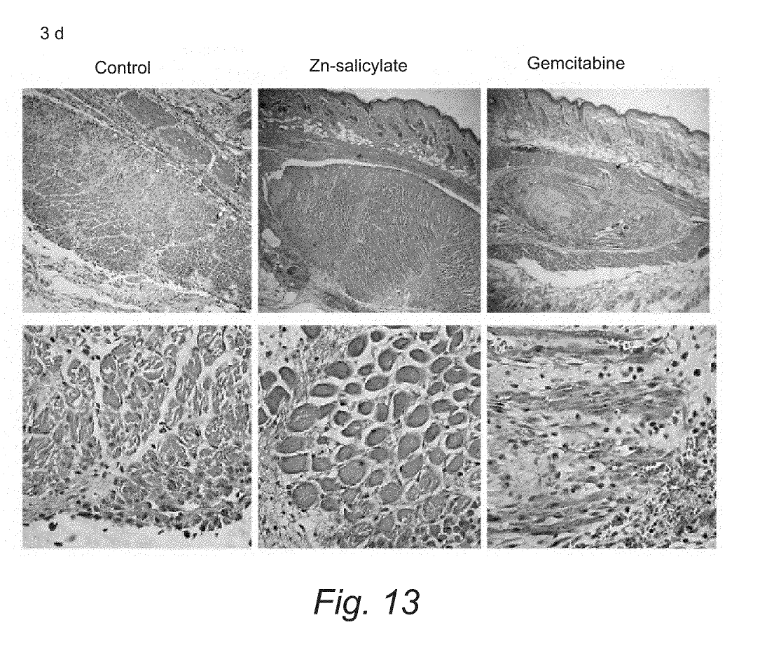

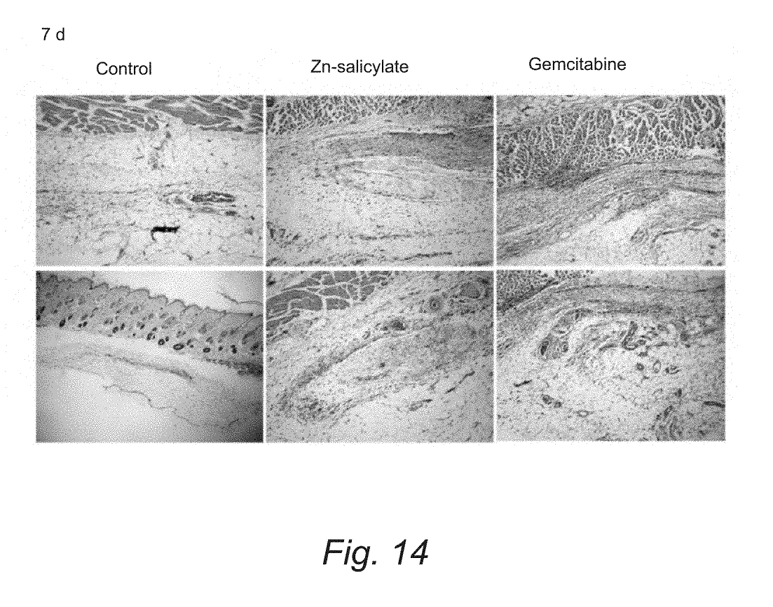

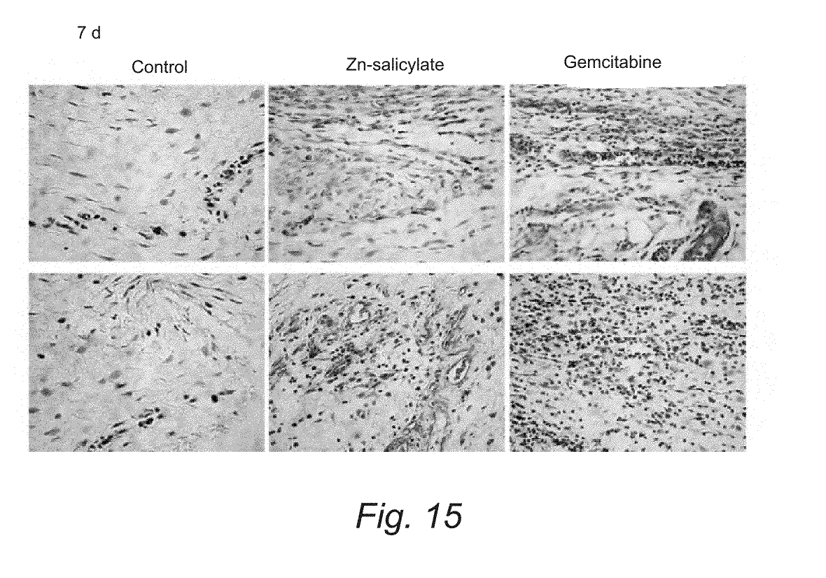

With reference now to FIGS. 10-15, the effect that microphages had on Zn-SA treated cells is illustrated. Three different fresh muscle tissue were cultured at 37° C. for 24 hours. One tissue was left untreated, the second included Zn-SA and the last one was treated with 100 (microM) of Gemitabine. Each were washed three times with PBS. 3×3×2 mm³ pieces of each tissue specimen were implanted subcutaneously into three different rats. At day 3, the control tissue showed disintegration and mild inflammation. The Gemcitabine treated tissue showed severe inflammation, where infiltrated blood vessels and penetrated lymphocytes were visible. Zn-SA treated tissue exhibited no visible of muscle structure. On day 7, the control tissue was completely disintegrated. Zn-SA treated tissue almost disappeared, where mild inflammation was shown and minimal amounts of capsulation. The stain for microphages were stained positive. The Gemcitabine treated group showed severe inflammation and fibrous capsules formed around the tissue. Microphages cleared Zn-SA fixed cells by phagocytosis without inflammation, both in vitro and in vivo. However, when the cancer cells were treated with chemotherapeutic drugs, such as gemcitabine, the cells broke into pieces (necrosis). These cells were difficult to clear, even when low concentrations of the drug were administered. The inflammation reaction was very severe in the in vivo implantation.

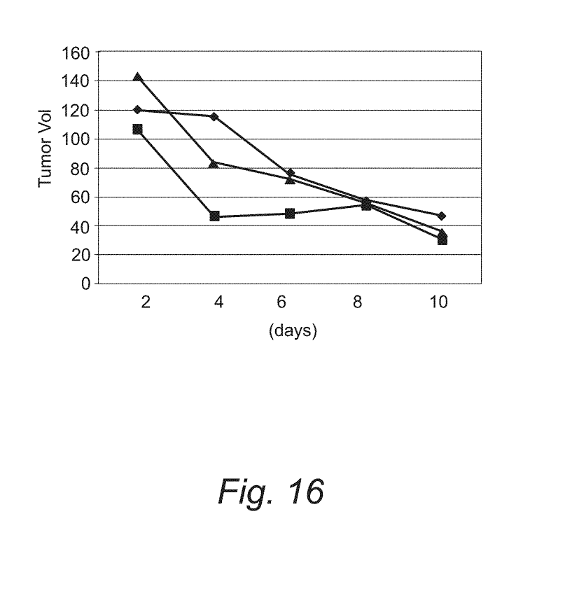

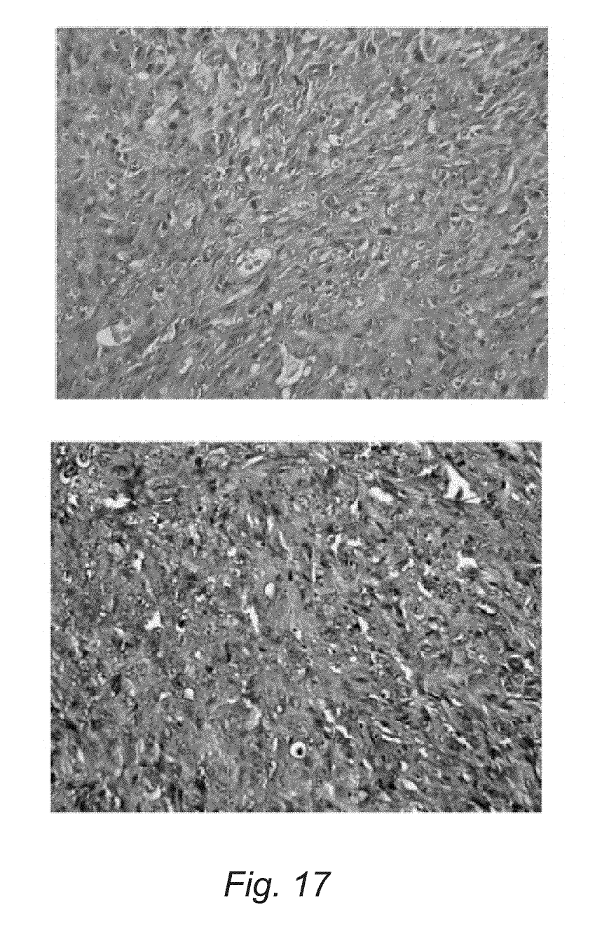

With reference now to FIG. 16, the results of an in vivo mouse study is illustrated. CFPAC, pancreatic cancer cells, were injected subcutaneously to induce xenograft tumor formation in a nude rat. The tumor grew over the course of 4 weeks; at which point 10 mg/kg of ZnSA was injected every other day. By the fourth day of treatment, the tumor significantly decreased. There were no adverse effects shown. In another embodiment, a topical treatment for osteosarcoma was given. Osteosarcoma SaoS-2 cells were injected subcutaneously to induce a xenograft tumor in a nude rat. The tumor grew over the course of 14 days; at which point 1% Zn-SA in 30% DMSO was topically applied. A decrease in tumor size and apoptosis were documented.

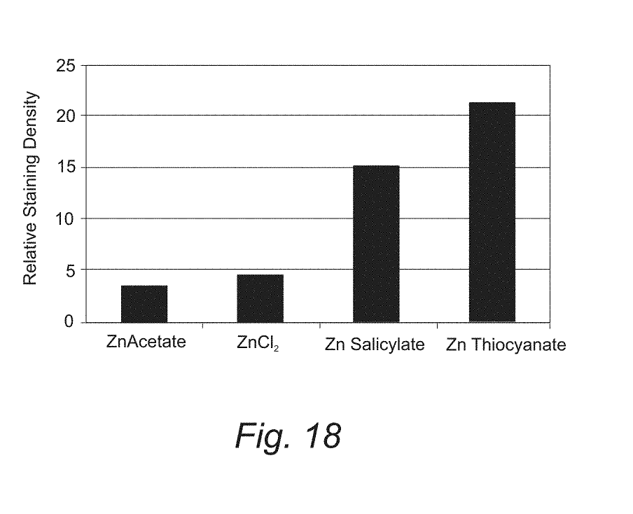

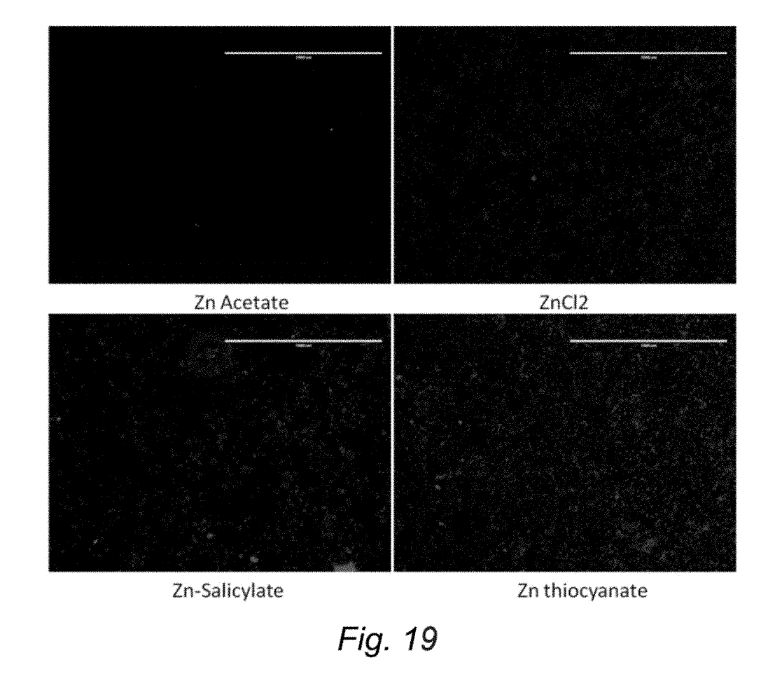

Referring to FIGS. 18 and 19, zinc transport in several complexes is illustrated. ZnSCN complexes show the lowest free Zn. The observation that Zn uptake is sigmoidal to SCN suggests that more than one SCN− molecule might be involved. There is also evidence that Zn transport may be involved with neutral species such as Zn-SA and Zn(SCN)₂.

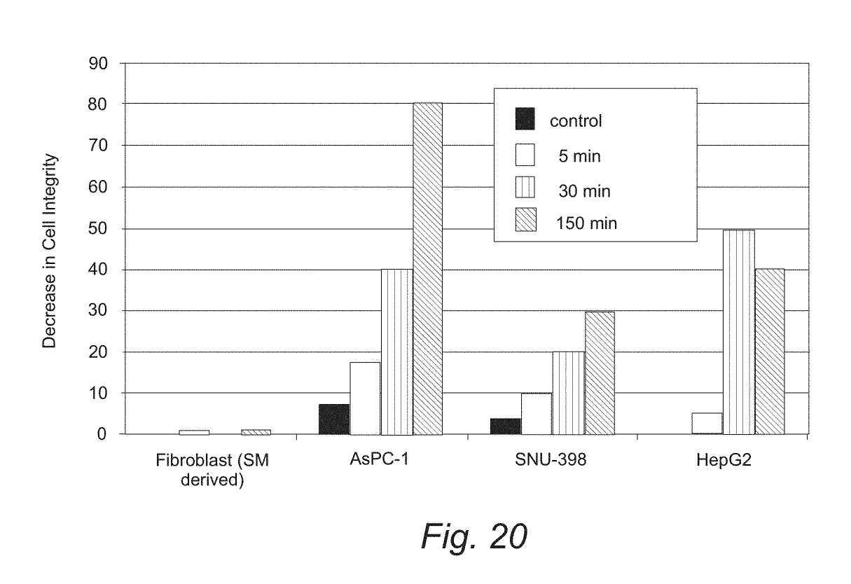

With reference to FIG. 20, cell proliferation rates of different liver cancer cells are illustrated over a 150-min period. The cancer cells treated with zinc thiocyanate showed a decrease in cell integrity and almost full survival rates.

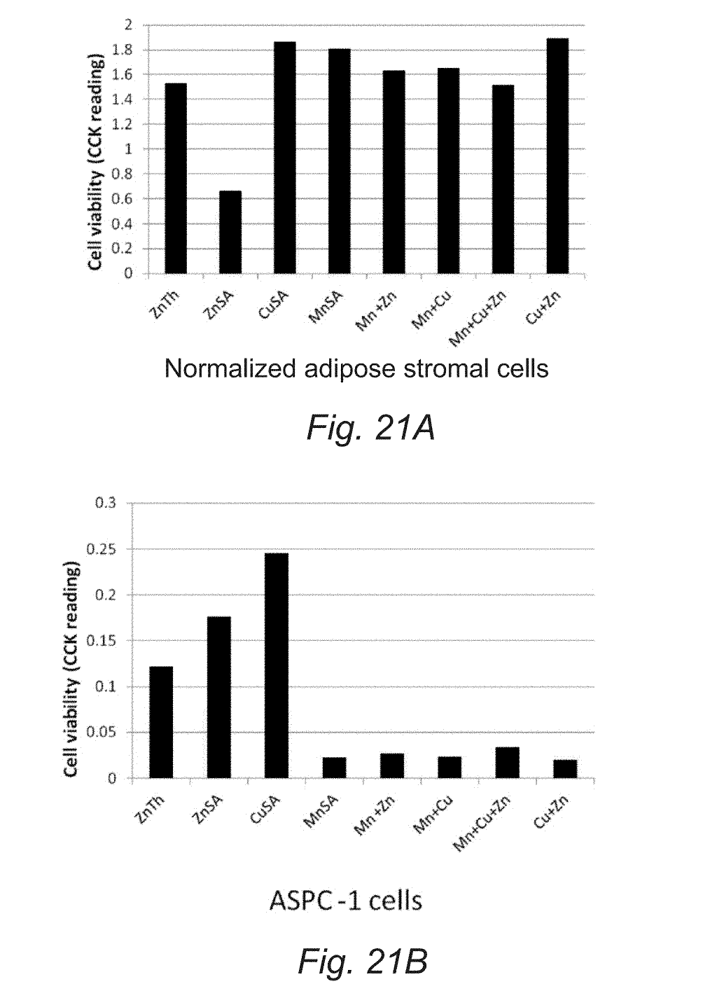

With reference to FIG. 21, a comparison of tumor cell and normal cell viability is illustrated. Zinc, copper, and manganese alone, or a combination thereof, inhibit cancer cell viability, but not normal cell function. The concentrations administered are as follows: ZnTh: 100 uM zinc thiocyanate, ZnSA: 100 uM Zinc salicylate 100 uM, CuSA: 100 uM Copric salicylate, MnSA: 100 uM manganese salicylate; MnGlu: 100 uM manganess gluconate; Mn+Zn: 50 uM MnSA+50 uM ZnSA; Mn+Cu: 50 uM MnSA+50 uMCuSA; Mn+Cu+Zn: 33.3 uM MnSA+33.3 uM CuSA+33.3 uM ZnTh.

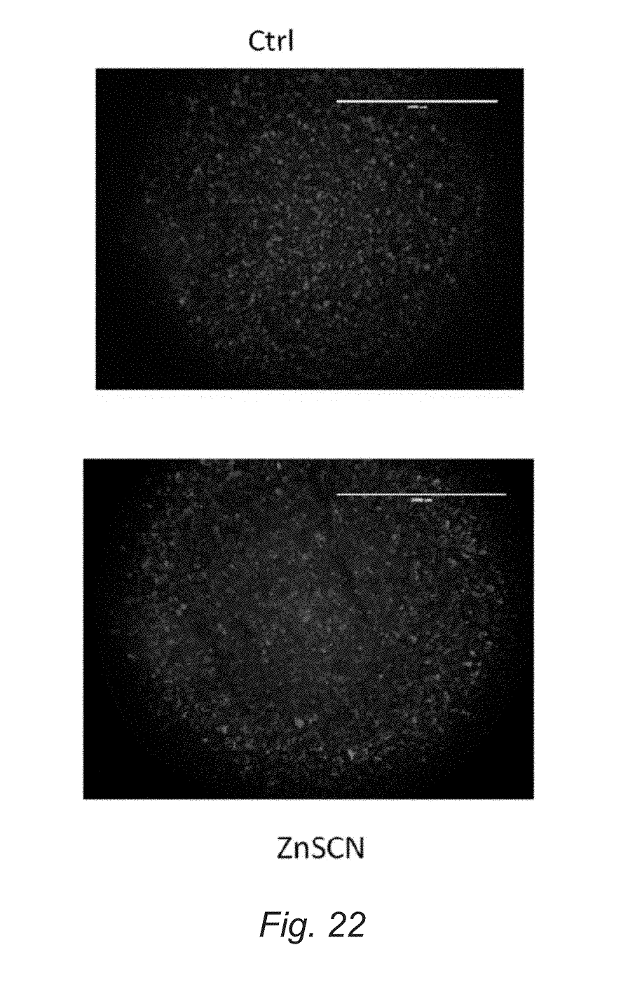

With reference now to FIG. 22, mitochondrial membrane potentials are illustrated as they relate to cancer cells. Cancer cells have hyperpolarized membrane potentials and zinc thiocyanate acts to depolarize this imbalance to rectify cell function. Cells were treated with zinc thiocyanate, zinc salicylate, manganese salicylate, and/or copper salicylate. The cells were stained with 1 μg/mL Rh-123 to be observed under a fluorescence microscope (excitation 450-490 nm; emission 515-565 nm).

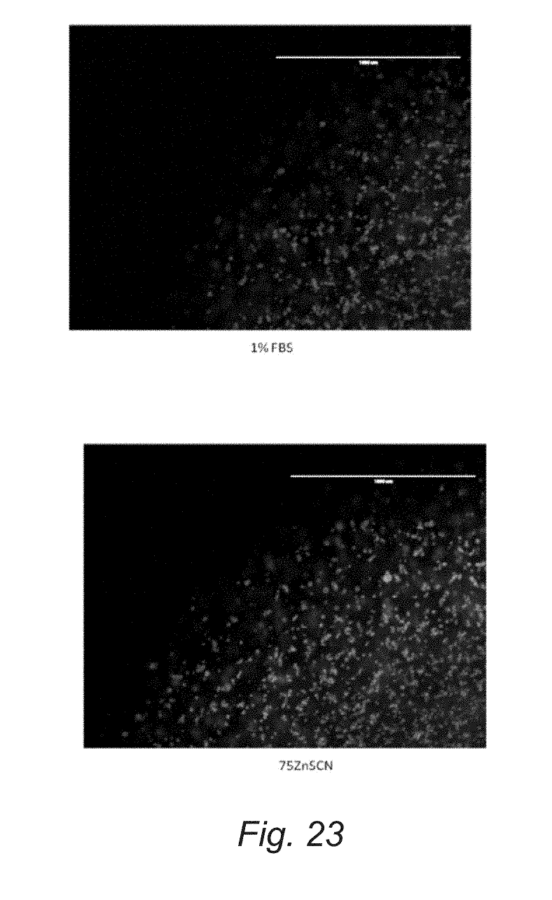

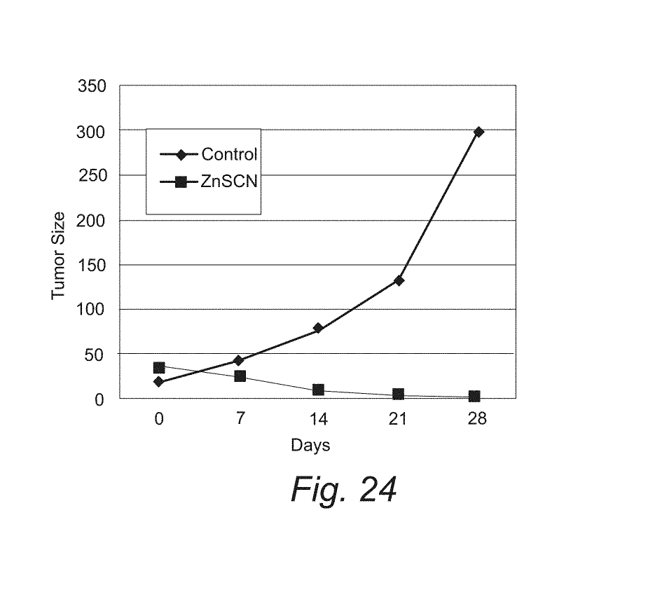

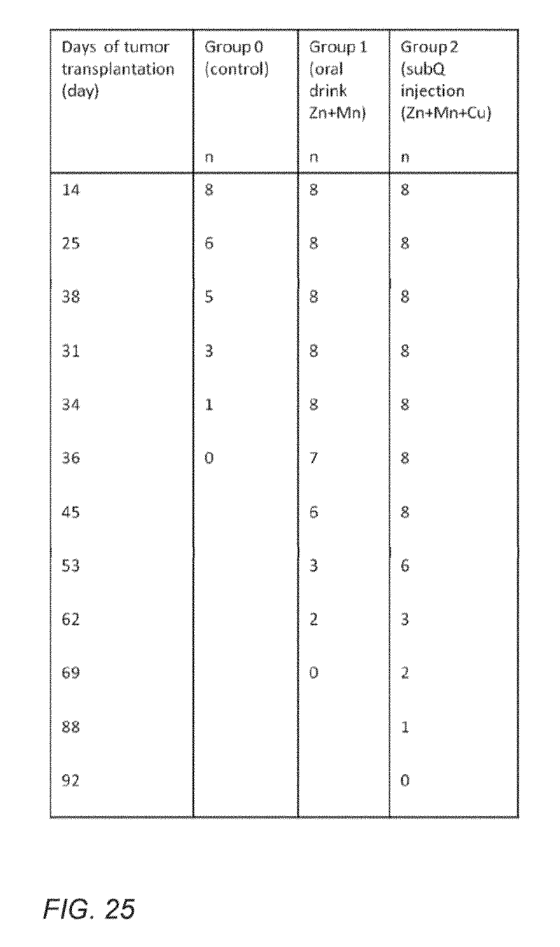

With reference now to FIG. 23, acidic vesicular organelles were detected. ZnSCN-treated cells were stained with 1 μg/mL acridine orange for 15 min. The samples were observed under a fluorescence microscope (excitation 546 nm; emission 575-640).

In another example, pancreatic cancer cells were injected to induce xenograft tumor formation in a rat. After two weeks, the tumor was treated with 10 mg/kg of zinc thiocyanate every other day for ten days. The tumor significantly decreased, and no adverse effects were observed.

Case Study 1:

A 66-year old man was diagnosed with stage IV non-small cell lung cancer (NSCLC) and showed signs of chest pains, spinal pain and extreme headaches. A computed tomography (CT) scan of his brain showed a tumor sized at 32 mm×29 mm. In addition, he had a 64-mm×53-mm tumor on his lungs. He refused anti-cancer therapy, but was put on pain medication (dexamethasone, phenytoin and morphine) to alleviate the symptoms. However, he continued to have pain. For 10 days, the patient received 100 mg of Zinc Thiocyanate, 25 mg of copper salicylate mixing with 1000 mL of sodium chloride 0.9% solution through infusion per day. On the 3^{rd} day of treatment, his headaches reduced; and by 10 days, his spinal pain disappeared. For the next 10 days, the dosage was reduced to 500 mL/day of the same solution. By the 16^{th} day, he no longer needed morphine to manage the pain and the dosage of dexamethasone and phenytoin was reduced. By the 20^{th} day, he no longer needed pain medication. He then began treatment orally; he ingested 50 mg of zinc thiocyanate and 10 mg of copper salicylate mixing with 100 mL of water 2 times/day for 40 consecutive days. His general health and quality of life improved in this time. On the 60^{th} day, a CT scan of his lungs and brain showed significant reduction of both tumors. The brain tumor decreased to 21 mm×17 mm and the lung tumor decreased to 24 mm×21 mm.

Case Study 2:

A 46-year old woman presented symptoms of headache and nausea. She had been treated for stage II breast cancer for the previous 16 months through surgery, chemotherapy and hormonal therapy. An MRI scan of her brain showed multiple metastases. The patient was given Dexamethasone tablets and Phenytoin for pain. The patient then began taking 125 mg of Zinc Thiocyanate and 30 mg of copper salicylate mixing with 1000 mL of sodium chloride 0.9% through in fusion daily through infusion. She reduced and eventually stopped the Dexamethasone use within 5 days. Her headache and nauseas symptoms were also controlled. The patient continued this dosage for another 12 days. She then took 3 dosages of 50 mg of Zn Thiocyanate and 10 mg of copper salicylate mixing with water daily. An MRI was taken 61 days after the start of treatment and showed a 70-90% reduction in metastases size.

Case Study 3:

A 58-year old female patient was diagnosed with stage IV breast cancer and invasive ductal carcinoma metastasized to multiple organs (lungs and brain). After refusing cancer treatment, she was treated with 100 mg of Zinc Iodide mixing with 1000 mL of sodium chloride 0.9% through infusion daily. Over the next 12 days, the patient's symptoms of coughing, headache and nausea gradually improved. She then took 3 dosages of 50 mg of zinc iodide per day orally. A chest ray was taken 61 days after the start of treatment, as shown the lung metastases decreased by more than 60%. A MRI scan showed that the brain metastases reduced by 50% also.

Case Study 4:

A 54-year old male smoker was suffering from long-term cough and chest pain. His symptoms included headache, nausea, difficulty with speech and movement and blurred vision. A mass was detected on the X-ray examination. CT scans of the chest and head confirmed lung cancer and multiple brain metastases, as well as brain edema. The patient was given Dexamethasone tablets and Phenytoin for pain. After refusing cancer treatment, he was treated with 100 mg of Zinc Thiocyanate mixing with 1000 mL of sodium chloride 0.9% solution though infusion daily. Within 7 days of treatment, all his symptoms were reduced. At this point, the dosage was decreased to 500 ml of the same solution through infusion per day for 10 days. By day 17, he began to take the treatment orally. The treatment included 3 dosages of 50 mg of Zinc Thiocyanate mixing with 100 mL of water orally per day for 40 days. At 63 days, a CT scan of the lungs showed a 50% decrease in tumor size. The brain metastases also decrease significantly.

Case Study 5:

A 26-year old female was diagnosed with an inoperable form of glioblastoma multiforme. Before the MRI was taken, she experienced severe headaches, nausea, paralysis on her left hand and face, and weakness in her left leg. She was treated with steroids and Temodal chemotherapy for a month. The patient was given Dexamethasone tablets and Phenytoin for pain. After short improvement, her symptoms became worse. The patient was treated with 125 mg of zinc iodide, 25 mg of Copper Salicylate, 25 mg of manganese salicylate mixing with 1000 ml of sodium chloride 0.9% and 10 ml of DMSO through infusion for 20 days. Within 14 days, she stopped taking pain medication. By the 20^{th} day, the patient's symptoms completely disappeared. For the next 40 days, she took 3 dosages of the 50 mg of Zinc Iodide, Copper salicylate 10 mg, Manganese salicylate 10 mg mixing with 200 ml of water/day. On day 62, a Mill scan showed a 60% reduction in brain tumor size and elimination of brain edema.

Case Study 6:

A 65-year old male had symptoms of weight loss and abdominal pain over a 2-month period. Blood test showed: total bilirubin at 78 mmol/Ll, GGT at 149 U/L, ALT at 187 U/L, AST at 113 U/L, and CA 19-9 at 385 U/L (normal range <37 U/mL). An ultrasound and CT suggested adenocarcinoma of the pancreatic head. At the head, there was a 38-mm×42 mm mass detected. In addition, his intrahepatic billiard tree was dilated. The patient was given 125 mg of Zinc Iodide mixing with 1000 ml of sodium chloride 0.9% and 10 ml of DMSO through infusion daily for 20 days. Within 7 days his pain completely disappeared. At 10 days, his blood test showed: total bilirubin at 56 mmol/Ll, GGT at 149 U/L, ALT at 73 U/L, AST at 73 U/L, and CA 19-9 at 184 U/L. The patient then took 3-50 mg dosages of Zinc Iodide per day, orally. He gained 4 kg of body weight. By the 63^{rd} day, the CT scan showed a decrease in tumor size to 12 mm×15 mm. There were no indications of liver metastases and his CA 19-9 level was 46 U/mL. The patient is symptom free.

Case Study 7:

A 71-year-old patient had prostate cancer with diffuse metastases, along with severe leg pain. He underwent hormonal therapy, until he developed severe bone pain. He took voltaren injections and solumedrol infusions to alleviate the pain. The patient's tests showed: Red Blood Cell (RBC) count at 3.2 million cells/uL, hemoglobin at 9 g/dL, serum calcium levels of 3.5 mmol/L, and prostate specific antigen (PSA) levels at 260 ng/mL. The patient was weak and lost 5 kg of weight in a month. He began the treatment with 125 mg of Zinc Iodide mixing with 1000 ml of sodium chloride 0.9% and 10 ml of DMSO through infusion. Within 7 days, his pain improved by 70%; and by day 20 he stopped all pain medication. His test showed: (RBC) count at 4.2 million cells/uL, hemoglobin at 10.7 g/dL, and serum calcium levels of 2.5 mmol/L. At this point, he took 3-50 mg dosages of Zinc Iodide mixing with 100 ml of water daily. His RBC, hemoglobin and serum calcium levels all normalized; and his PSA decrease dramatically to 40 ng/mL. His quality of life improved greatly, and he had no symptoms. Though he decided not to take any tests for bone metastases, his ultrasound and x-ray scans revealed no abnormalities.

Case Study 8:

A 14-year-old girl was diagnosed with lymphoblastic leukemia and underwent chemotherapy with no success. She developed chemotherapy-induced bone marrow aplasia, along with severe anemia, thrombocytopenia, and leukocytopenia. Her peripheral blood blast count was 72% and she had systematic fungal infections, high fever, bleeding diarrhea, and pneumonia. The patient was treated with 100 mg of Zinc Iodide and 25 mg of copper salicylate mixing with 1000 ml of sodium chloride and 10 ml of DMSO for 10 days through infusion. She was then treated with the same dosage every 2 days, for 20 days. Within 20 days, her stool and fever were normal. Her blood count levels dramatically improved also. She no longer needed blood and thrombo-mass transfusions. Her peripheral blood blast count decreased to 16%. Within 30 days, she no longer had diarrhea, fever, pneumonia or signs of systematic fungal infections. The patient then took 30 mg of Zinc Iodide and 10 mg of copper salicylate mixing with 100 ml of water 3 dosages per day for 90 days, a biopsy in 120 days from the beginning of the treatment showed complete remission of acute lymphoblastic leukemia.

Case Study 9:

A 66-year old man presented with jaundice, abdominal discomfort and lack of appetite. He could not eat or drink properly. Over the last 3 months, his weight decreased by 8 kg. CT scans showed an intrahepatic cholestasis. There was also a hypodense lesion with an intraductal growth type, sized at 3.6 cm×4.1 cm, on his liver. An ultrasound showed liver tissue hardening associated with liver cirrhosis. He was diagnosed with cholangiocarcinoma. His blood test showed: total bilirubin at 139 mmol/Ll, GGT at 1410 U/L, GOT at 163 U/L, GPT at 127 U/L, and CA 19-9 at 78 U/L. The patient was sent home with palliative treatment and given 3-6 months to live. The patient was treated through infusion with 200 mg of zinc Zinc iodide mixing with in 1000 mL of 0.9% sodium chloride and 10 mL of DMSO for 20 days. Within 5 days, his digestive health improved significantly. After 10 days, his blood test showed: total bilirubin at 75 mmol/Ll, GGT at 1267 U/L, GOT at 121 U/L, and GPT at 97 U/L. At 21 days, his jaundice improved, and his blood test showed: total bilirubin at 56 mmol/Ll, GGT at 978 U/L, GOT at 85 U/L, and GPT at 193 U/L. The patient then took 3-50 mg dosages of Zinc Iodide/day in capsule form for 3 months. 105 days after the beginning of treatment, the patient no longer experienced symptoms and had gained 4 kg of weight. The ultrasound showed a tumor decrease to 1.8 cm×2.1 cm, with remarkable improvements to the bile duct and cholestasis. The blood tests showed: total bilirubin to 28 mmol/L, GOT levels at 67 U/L, GPT levels at 54 U/L and GGT levels at 784 U/L.

Case Study 10:

A 62-year-old man was diagnosed with esophageal cancer with multiple lymphatic metastases. His blood chemistry profile was normal, but his CEA levels were 6.7 ng/mL. The patient presented symptoms of extreme fatigue, difficult swallowing, chest pains, fever and weight loss. An endoscopy reveled a 4.2 cm×3.7 cm lesion on his esophagus. The tumor had poorly differentiated adenocarcinoma. A CT suggested multiple lymphatic metastases. The patient underwent treatments of 200 mg of zinc iodide mixing with 1000 mL of 0.9% sodium chloride solution and in 10 mL of DMSO, 6 times/week for 4 weeks. After 3 days, the patient's general health improved, with no symptoms of fever or pain. He also stopped steroid treatment. Within 3 weeks, he could eat and drink normally. After 24 days of the infusion treatment, he began taking 50 mg Zinc Iodide capsules, 3 times/day for 90 days. By the 105^{th} day, the tumor decreased to 1.5 cm×1.4 cm and the lymph nodes were clear of metastases.

Case Study 11:

A 69-year old man presented signs of hoarseness, difficulty swallowing, and weight loss over a 5-month period. He also had ear, nose and neck pain. A laryngoscopy showed a tumor on his larynx, sized at 3.5 cm×3.6 cm. A physical examination showed enlargement of his lymphatic glands also. He was treated with 100 mg of zinc thiocyanate mixing with 1000 mL of 0.9% sodium chloride and 10 mL of DMSO for 20 days. His pain disappeared in 7 days and all his symptoms improved by 10 days. The patient was then given 3-50 mg Zn Thiocyanate capsules daily for 90 days. By the 100^{th} day, the patient was relieved of physical symptoms and his lymphatic glands reduced in size. The lymphatic tumor decreased to 0.8 cm×1.2 cm.

Case Study 13:

A 74-year-old man had history of smoking and was diagnosed with COPD. The patient presented with symptoms of chest pain, fatigue, difficulty breathing for 2 months prior to treatment. A CT scan depicted a 5.8 cm×5.6 cm mass on his left lung; he was diagnosed with non-small cell adenocarcinoma. The patient was treated with 3-50 mg Zn Thiocyanate capsules/day for 100 days. Within 10 days, his symptoms began to improve. 96 days after his first treatment, he reported dramatic improvement in general health and breathing capabilities. A CT scan showed a reduction in the tumor to 2.7 cm×3.1 cm.

Case Study 14:

An 81-year-old man presented with cough and chest pains that persisted over 6 months. In that time, he also lost 6 kg of weight. In the last 20 days, the patient developed hemoptysis, which lead him to hospitalization. A CT scan of his chest, abdomen and lower pelvic regions revealed a 5.7 cm×6.1 cm mass in the superior right hilum. He also had total opacification of the right lung. The patient was diagnosed with lung cancer and sent home. The patient was treated with 3-50 mg Zinc Iodide capsules/day for 120 days. After 5 days, his breathing capabilities and cough improved. By 12 days, he no longer had blood in his sputum. After 121 days of improved overall health, a CT scan showed 80% clearance of opacification and a reduced tumor at 2.3 cm×2.9 cm.

Case Study 15:

A 68-year old male had a history of COPD and infective bronchitis. Due to this, he was hospitalized 4-5 times/year. For 3 years, he used a corticosteroid inhaler, bronchodilator, antibiotics and COPD medication to no avail. He was also taking hypertension medication as a result of the COPD treatment. His blood pressure was 160/96 and Forced Expiratory Volume (FEV₁) at 58%. The patient was treated with 3-50 mg Zn Iodide capsules/day. After 3 days, he no longer needed fast acting bronchodilator inhalers. Within 10 days, he no longer needed corticosteroid inhalers either. At 30 days, his physical examination showed remarkable improvement of his COPD related symptoms. His (FEV₁) was 67% and his blood pressure decreased to 130/90. In the following 2 months, he did not have any COPD related episodes. Even though he was not taking COPD medication, there were no signs of infections either. 92 days after his first Zinc iodide treatment, he no longer had shortness of breath, his (FEV₁) was 75% and his blood pressure decreased to 125/90.

Case Study 16:

A 47-year old man presented symptoms of abdominal pain, nausea, and vomiting. A computed tomography (CT) scan of the abdomen showed a 5.6×6.3 tumor on the pancreatic gland. He also had multiple metastases on his liver. He was diagnosed with pancreatic adenocarcinoma. His CA 19-9 (levels of tumor-associated antigens in the blood) was 365 U/mL. The patient was treated through infusion with 200 mg of zinc Iodide mixing with 1000 mL of 0.9% sodium chloride solution and 10 mL of DMSO. The symptoms presented improved after 2 days of treatment. By the 5^{th} day, the patient was almost symptom-free; and he could eat soft foods. Over the next 20 days, his quality of life of improved and by day 30 he could eat and drink normally. After 35 days on the treatment, an ultrasound was taken of the pancreas. The tumor had decreased to 4.3 cm×5.2 cm. His CA 19-9 levels decreased to 168 U/mL. After 40 treatments, the patient began treatment orally; he was given 50 mg zinc iodide capsules 3 times/day. After 155 days on the oral treatment, a CT of his pancreas was taken. The tumor decreased to 2.4 cm×3.6 cm. The liver metastasis decreased by 50% also.

Case Study 17:

A 58-year old man with chronic hepatitis C presented symptoms of abdominal pain. He had a history of liver cancer and liver cirrhosis. He had also been diagnosed with type 2 diabetes three years prior. The CT scan revealed a hypodense lesion, sized at 8.5 cm×7.9 cm, on the right of his liver. Laboratory results on known cancer markers showed elevated fetoprotein (AFP) levels at 1450 IU/L, total protein levels of 6.2/dL, albumin levels at 3.2 g/dL, GOT levels at 326 IU/L, GPT levels at 293 IU/L and GGT levels at 251 IU/L. His blood glucose level was at 8.5 mmol/L. The patient was treated through infusion with 200 mg of zinc iodide mixing with in 1000 mL of 0.9% ml of sodium chloride. Within 5 days his abdominal pain disappeared, and his general health improved. After 10 days of treatment the patient showed reduction in all cancer related markers. The levels include AFP: 1209 IU/L, GOP: 224 IU/L, GPT: 187 IU/L, and GGT: 93 IU/L. The patient's blood glucose level was 7.6 mmol/L. After 21 days, the ultrasound showed a decrease in tumor size on the liver, sized at 6.3 cm×5.4 cm. His cancer related markers included: AFP: 678 IU/L, GOP: 146 IU/L, GPT: 126 IU/L, and GGT: 124 IU/L. The patient's blood glucose level was 6.7 mmol/L. For the next 90 days, the patient took 50 mg of zinc iodide orally in capsule form 3 times/day. A CT scan was then taken, revealing a decreased tumor, sized at 2.6 cm×2.1 cm. His cancer related markers included: AFP: 107 IU/L, GOP: 195 IU/L, GPT: 69 IU/L, and GGT: 115 IU/L. The patient's blood glucose level was 7.4 mmol/L.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method for treating cancer, chronic obstructive pulmonary disease, asthma, diabetes, sickle cell disease, thalassemia, or any other inflammatory disease, and/or promoting healing thereafter, the method comprising:
identifying a subject afflicted with cancer, chronic obstructive pulmonary disease, or an inflammatory disease; and
administering a therapeutically effective dose of a pharmaceutical composition comprising at least one metal-containing compound, the at least one metal-containing compound comprising a metal selected from the group consisting of zinc, copper, manganese, and combinations thereof.

2. The method of claim 1 wherein the at least one metal-containing compound has formula by MXₙ wherein M is zinc, copper, manganese ions, X is a counter ion, and n is the number of X to maintain charge neutrality.

3. The method of claim 1 wherein X is sulfate, O, S, halide, gluconate, ascorbate, lactate, laurate, peroxide, salicylate, thiocyanate, aspartate, or benzoate.

4. The method of claim 1 wherein the at least one metal-containing compound includes at least one a salicylate and/or at least one gluconate.

5. The method of claim 1 wherein the pharmaceutical composition includes a sulfur-containing anion, metal-containing compound including the sulfur-containing anion or the pharmaceutical composition further comprising a second compound including the sulfur-containing anion.

6. The method of claim 5 wherein the sulfur-containing anion comprises thiocyanate, isothiocyanate, thiosulfate, or methionine.

7. The method of claim 5 wherein the pharmaceutical composition comprises zinc thiocyanate.

8. The method of claim 5 wherein the at least one metal-containing compound includes at least one a salicylate and/or at least one gluconate.

9. The method of claim 5 wherein the at least one metal-containing compound is zinc iodide.

10. The method of claim 1 wherein the pharmaceutical composition further comprises a fluid carrier biocompatible with human tissues.

11. The method of claim 10 wherein the fluid carrier further comprises physiological strength electrolytes.

12. The method of claim 10 wherein the fluid carrier further comprising additive ingredients selected from the group consisting of minerals, medical solvents, ethanol, coloring agents, antimicrobial preservatives, blending agents, and combinations thereof.

13. The method of claim 10 wherein the fluid carrier further comprises a surfactant in a concentration of about 0.05 to 2%.

14. The method of claim 1 wherein a dose of the pharmaceutical composition is from 60 to 200 mg administered orally over a 24-hour period.

15. The method of claim 1 wherein a dose of the pharmaceutical composition is from 0.5 to 10 mg/kg subject body weight administered parenterally over a 24-hour period.

16. The method of claim 1 wherein a dose of the pharmaceutical composition is 10 to 1000 mg administered topically or by medicinal electrophoresis.

17. The method of claim 1 wherein the pharmaceutical composition further comprises a copper-containing compound.

18. The method of claim 1 wherein the metal-containing compound is a metal salicylate complex selected from the group consisting of zinc salicylate, copper salicylate, manganese salicylate, and combinations thereof.

19. The method of claim 18 wherein the metal salicylate complex includes zinc salicylate.

20. The method of claim 18 wherein the at least one metal-containing compound includes at least one salicylate and/or at least one gluconate.

21. The method of claim 18 wherein a dose of the metal salicylate complex is 10 to 1000 mg of metal salicylate/50 kg of body weight per day.

22. The method of claim 18 wherein a dose of the metal salicylate complex is 0.04 to 10 mg of metal salicylate/kg of patient body weight per day.

23. The method of claim 18 wherein the metal salicylate complex includes zinc salicylate, copper salicylate, manganese salicylate, and succinic acid.

24. The method of claim 1 wherein the at least one metal-containing compound is an aspirinate-metal complex selected from the group consisting of zinc aspirinate, copper aspirinate, manganese aspirinate, and combinations thereof.

25. A pharmaceutical composition for treating cancer, chronic obstructive pulmonary disease, or an inflammatory disease, the pharmaceutical composition comprising;
at least one metal-containing compound, the at least one metal-containing compound comprising a metal selected from the group consisting of zinc, copper, manganese, and combinations thereof.

26. The pharmaceutical composition of claim 25 wherein the metal-containing compound includes a sulfur-containing anion or the pharmaceutical composition further comprises a second compound including the sulfur-containing anion.

27. The pharmaceutical composition of claim 26 wherein the sulfur-containing anion comprises thiocyanate, isothiocyanate, thiosulfate, or methionine.

28. The pharmaceutical composition of claim 25 wherein the at least one metal-containing compound includes at least one salicylate and/or at least one gluconate.

29. The pharmaceutical composition of claim 25 wherein the metal-containing compound is a metal salicylate complex selected from the group consisting of zinc salicylate, copper salicylate, manganese salicylate, and combinations thereof.

30. The pharmaceutical composition of claim 29 further comprising succinic acid.

31. The pharmaceutical composition of claim 25, wherein the at least one metal-containing compound is an aspirinate-metal complex selected from the group consisting of zinc aspirinate, copper aspirinate, manganese aspirinate, and combinations thereof.

32. The pharmaceutical composition of claim 25 wherein the at least one metal-containing compound is zinc iodide.

33. The method of claim 1 wherein the at least one metal-containing compound is delivered as a nanoparticle.

34. The method of claim 32 wherein the at least one metal-containing compound is zinc sulfide.
